(19) United States
(12) Patent Application Publication
(10) Pub. No.: US 2021/0068695 A1
(43) Pub. Date: Mar. 11, 2021

(54) Method Providing ECG Analysis Interface and System

(71) Applicant: Peacs Investments B.V., Nieuwerbrug aan den Rijn (NL)

(72) Inventors: Peter Michael van Dam, Nieuwerbrug aan den Rijn (NL); Eelco Matthias van Dam, Nieuwerbrug aan den Rijn (NL)

(21) Appl. No.: 17/050,695

(22) PCT Filed: Apr. 25, 2019
(86) PCT No.: PCT/NL2019/050244

§ 371 Date: Oct. 26, 2020

(30) Foreign Application Priority Data
Apr. 25, 2018 (NL) 2020827

(51) Int. Cl. A61B 5/0468; G16H 40/63; G16H 10/60; G16H 50/70; G16H 15/00; G16H 50/30; G16H 50/50; G16H 40/40; A61B 5/04; A61B 5/044; A61B 5/0472; A61B 5/00; G06T 11/00

(52) U.S. Cl. CPC A61B 5/0468; G16H 40/63; G16H 10/60; G16H 50/70; G16H 15/00; G16H 50/30; G06T 2210/41; G16H 40/40; A61B 5/04012; A61B 5/044; A61B 5/0472; A61B 5/7275; G06T 11/00; G16H 50/50

(57) ABSTRACT

The present application relates to a method, such as implemented on a computing device part of and/or coupled to an EGG device, for providing an EGG analysis interface relating to a person, the method comprising steps of: —obtaining data entry identifying the person for obtaining historical data pertaining to at least one preceding data point from a database, —obtaining at least one historical EGG measurement result pertaining to said at least one data point from the database, —obtaining historical EGG electrode location information relating to the electrode placement of the respective at least one data point, —obtaining latest or new EGG electrode location information measurement of electrode positions for a live EGG measurement, —obtaining latest or new EGG measurement results of the person with the location measured electrodes, —performing steps of verification as to differences between the historical EGG electrode location information and the latest or new EGG electrode location information, —assembling the EGG analysis interface comprising a representation of differences between the at least one historical EGG measurement result and the latest or new EGG measurement results, preferably if the steps of verification provide a sufficient result.

The present invention relates to a method, such as implemented on a computing device part of and/or coupled to an ECG device, for providing an ECG analysis interface towards a person. Furthermore, the present invention relates to a system for performing such a method.

Over the last decades, a variety of devices has been developed and improved in order to obtain and provide relevant medical information regarding the heart and torso of a person in order to diagnose disorders or ailments of the heart and cardiovascular system.

ECG devices have been devised in order to provide information relating to the electrical functioning of the heart and other functions of the heart identifiable by such means. Echo imaging devices have been used to provide visible information relating to the functioning of the heart in the torso.

Several decades ago, such as around the 70s, the ECG technology provided much improved systems with relatively low noise and was as such recognized as a valuable data source pertaining to the functioning of the heart. This was before major quality improvements of imaging technologies, such as MRI, CT and sound echo. However, these imaging technologies have improved in major ways at least since the 90s. Because of these developments in imaging technologies, the relevance of the ECG technology as decreased or stabilized as the imaging technologies provided more and more detailed information.

It is an object of the present invention to provide a very cost-effective identification of ailments or conditions, or early stages thereof. To this end, the present invention provides a method, such as implemented on a computing device part of and/or coupled to an ECG device, for providing an ECG analysis interface relating to a person, the method comprising steps of:
obtaining data entry identifying the person for obtaining historical data pertaining to at least one preceding data point from a database,
obtaining at least one historical ECG measurement result pertaining to said at least one data point from the database,
obtaining historical ECG electrode location information relating to the electrode placement of the respective at least one data point,
obtaining further, latest or new ECG electrode location information measurement, such as of electrode positions for a live ECG measurement,
obtaining respective ECG measurement results of the person relating to the further, latest or new location measured electrodes,
performing steps of verification as to differences between the historical ECG electrode location information and the latest or new ECG electrode location information,
assembling the ECG analysis interface comprising a representation of differences between the at least one historical ECG measurement result and the latest or new ECG measurement results, preferably if the steps of verification provide a sufficient result.

A first advantage of such a method according to the present invention is that a comparison is provided, in a reliable manner, regarding two or more ECG measurements, each taken at a different point in time with distinct electrode placements. ECG measurements with the same electrode placement during the same measurement session are very comparable. The reason for this is that the signals are based on the same electrode placement and does interact with the torso and the heart in the same way verifiable by the control electronics of the ECG electrodes.

However, ECG measurements relating to separate measurement sessions have been useful as to each measurements per se, but comparability was low or unreliable. According to the prior art, markings on the body, such as by tattooing or similar inking, have been used in order to provide repeatable results of the ECG measurements in order to compare the ECG measurements. However, such markings are not desirable during the timeframe of months or years of treatment nor thereafter. Therefore, such markings basically never left the realm of basic research. Such disadvantages, combined with the availability of the said imaging technologies, such as MRI, CT and sound echo providing a lot of information and being rather more fashionable in the medical world have led attention away from further developing the use of ECG other then performing basic measurements.

The present invention provides a reliable way to compare ECG measurements between distinct measurements sessions, such as with a time interval of days, weeks, months, quarters, years, or even more. An insight of the present inventor is that ECG results can be used for diagnosis or recommendations of conditions that occur with certain conditions such as the presence of certain genes in the genome of a person. In such cases, even persons having the predetermined combination of genes for an ailment might not develop such conditions at all, in the same manner or at the same age. Therefore, monitoring persons with such genes with expensive imaging technologies is inefficient.

The present invention provides the advantage of being able to monitor identify persons by means of ECG monitoring with repeatable electrode placement leading to possibilities of identifying early onset of such conditions with a high level of discernibility with the further advantage of low intrusiveness of the procedure. It will be shown below, and it is established, that identification of such early onset is achieved by applying the present invention, even when the most modern imaging equipment would not detect such early onset.

An example thereof is for example the condition of arrhythmogenic right ventricular cardiomyopathy (ARVC). Such condition consists of the onset of a delayed activation region in the heart, an outer wall or septum thereof. In the example below indicates how ECG analysis, such as waveform analysis, provides information as to differences of waveforms as recorded in separate ECG measurement sessions. However, the example also shows that such differences in waveforms would also occur when one or more electrodes are placed in a different position between such separate ECG measurement sessions. Therefore, it has not been possible to use ECG measurements for such purposes. ECG has been used for determining issues regarding rhythm of the heart, and for determinations based on the timing of certain ECG intervals, such as a PQ time or a QRS integral. However, waveform analysis relating to the shape of the waveform, or an analysis of the waveform of repolarization has not been available without the present invention.

In the context of this description, latest means measurements made during a new, latest or later instance of measuring an ECG and/or electrode location, preferably for storage as a new data point to be referred to as one of the at least one preceding data point for reference with a measurement at a later point in time, such as after a day, week, month, trimester, year or other, then being the current new measurement.

According to a first preferred embodiment, the representation of differences comprises a waveform representation of the historical ECG measurement results and of the further, latest and/or new ECG measurement results. Such waveform representation, such as plotting of the waveform on a display, provides the indicated advantages of being able to compare waveforms between ECG measurements of different ECG measurement sessions.

According to a further preferred embodiment, the method comprises steps of aligning the respective waveform representations, preferably aligning the respective waveform representations in relation to a time axis of an ECG graph. This allows for identifying differences of waveforms at similar points in time during the polarization/repolarization cycle.

Further preferably, such steps of aligning the respective waveform representations are based on respective RMS signals. RMS signals are calculated based on measurement information from the electrodes of the ECG measurements. An advantage there of is that a relatively straightforward process to align RMS signals from distinct ECG measurement sessions is provided due to the shape of such RMS signal.

Further preferably, alignment is based on the peak of the QRS complex, preferably based on the peak of the QRS complex according to the RMS signal. Such embodiments provide very advantages alignment of the information coming from distinct ECG measurement sessions.

Because the present invention or embodiments thereof provide the advantage of having the ECG electrodes of the distinct ECG measurement sessions over time substantially the same or within predetermined threshold deviations, differences between the ECG signals, preferably as aligned are provided in a readily visible way. Advantageously, differences that would have been caused by this similar placement of electrodes are eliminated when such differences between the ECG signals are determined.

According to a further preferred embodiment, the method comprises steps of providing the ECG analysis interface, preferably a representation of differences thereof, with highlighting information based on a predetermined set of criteria. Such highlighting of information is envisioned to be provided in several ways, such as encircling, or similar, of such differences or changing the color of the waveform at the location of such differences.

Further preferably, the predetermined criteria are based on a recognition of relevant differences relating to at least one condition or ailment. Based on relevant information as to known variations in waveforms due to certain conditions, such wave form variations can be determined automatically and indicated as per such embodiments. Also, numerically discernible indicators that are less easy to show in the waveform can be highlighted as such. Further way of indicating such differences is to provide an enlargement of such parts of the waveform that are identifiable.

Examples of a condition or ailment to be identified by the present invention comprise a arrhythmogenic cardiomyopathy (ACM), sudden cardiac death risk (SCD), arrhythmogenic right ventricle cardiomyopathy (ARVC), or initial stages of PVC development.

According to a further preferred embodiment, the representation of differences comprises at least one numerical representation of the differences, preferably thereby indicating at least one respective biomarker.

A further aspect according to the present invention or aspect relates to a method, such as implemented on a computing device part of and/or coupled to an ECG device, for providing an ECG analysis interface relating to a person, the method comprising steps of:
obtaining data entry identifying the person for obtaining historical data pertaining to at least one preceding data point from a database,
obtaining historical ECG electrode location information relating to the electrode placement of the respective at least one data point,
obtaining an optical, preferably 3D, recording of the torso of the person and/or latest or new ECG electrode location information measurement of an initial placement of positions of the ECG electrodes for a live ECG measurement,
rendering or assembling feedback information for providing a feedback signal based on the historical ECG electrode information of the respective at least one data point,
visualizing of the feedback signal relative to the torso of the person.

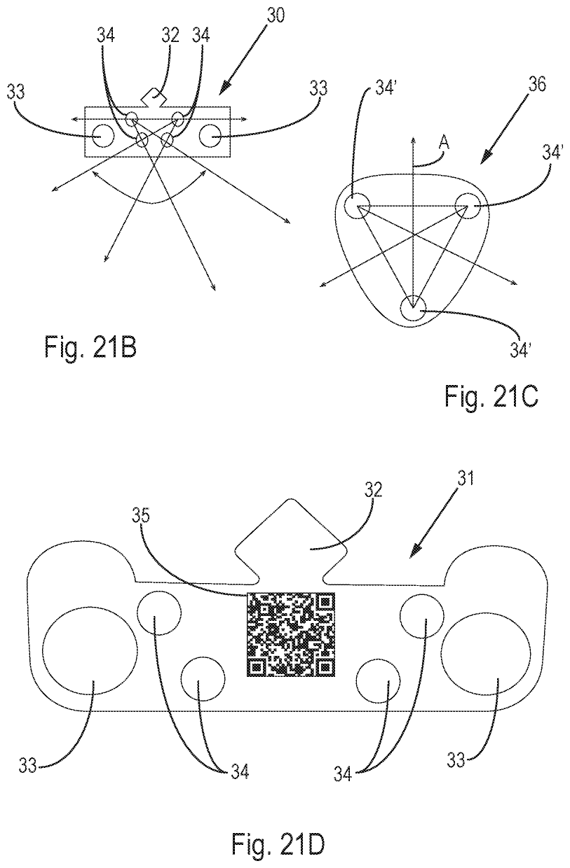

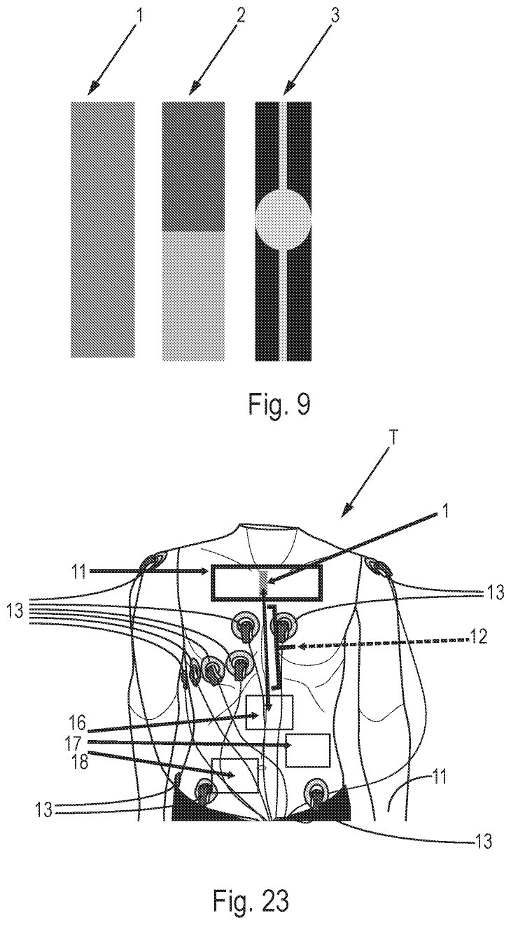

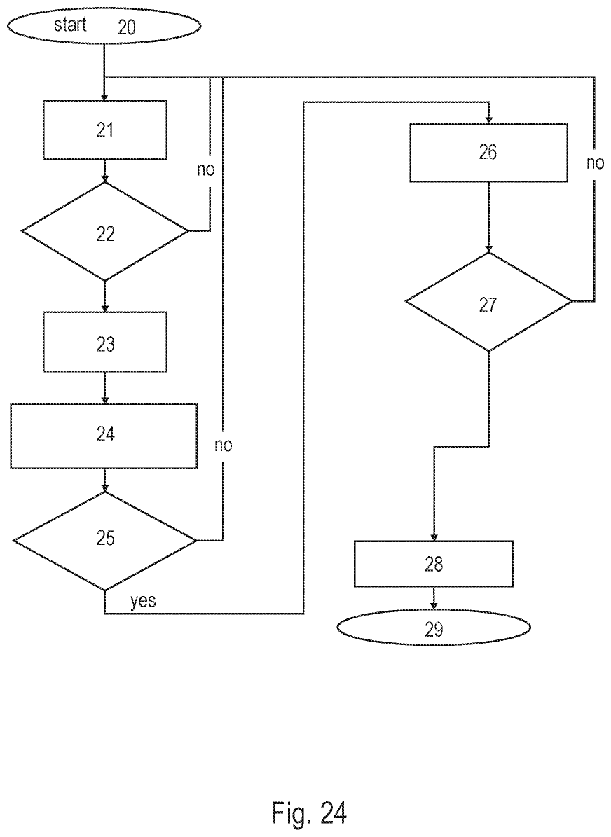

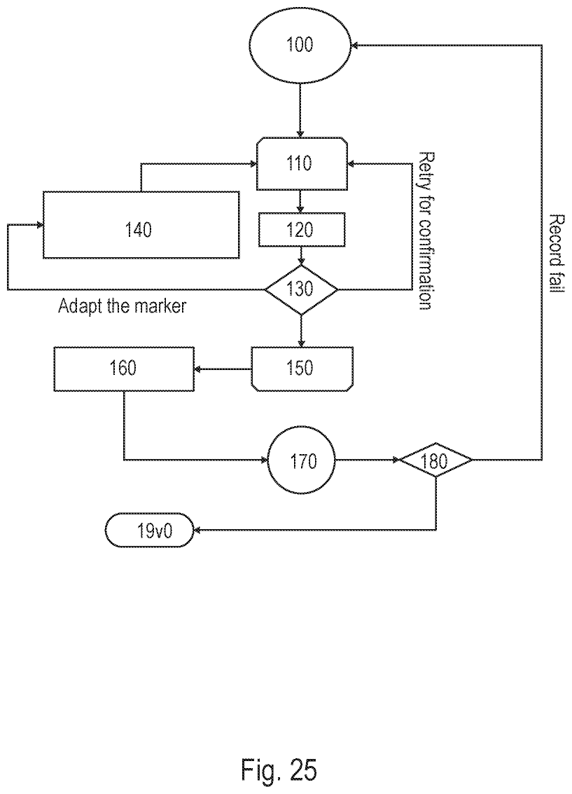

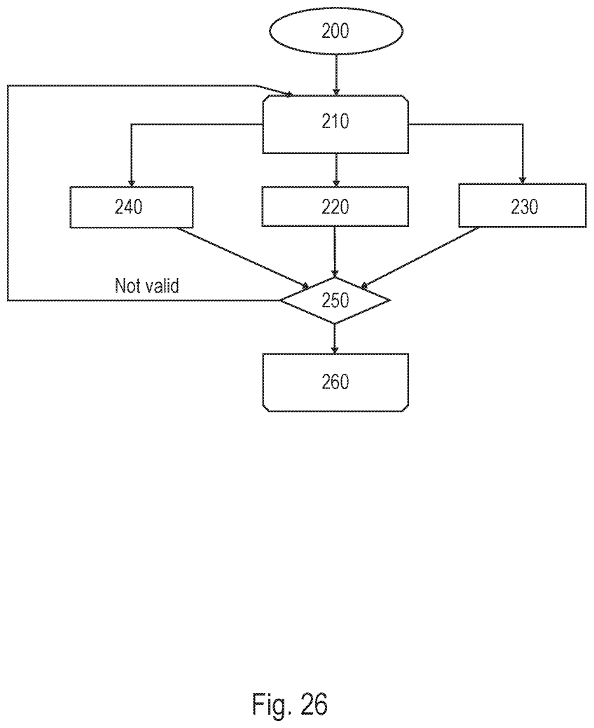

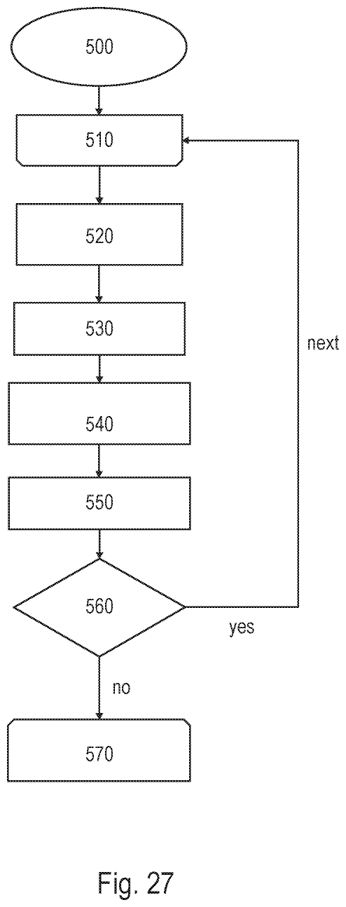

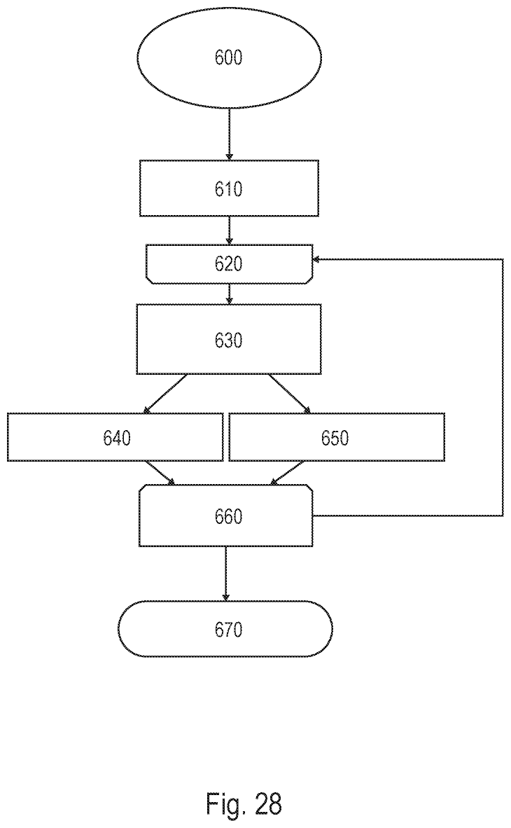

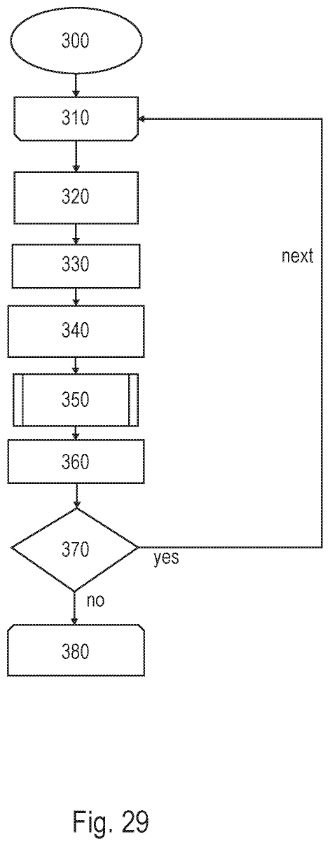

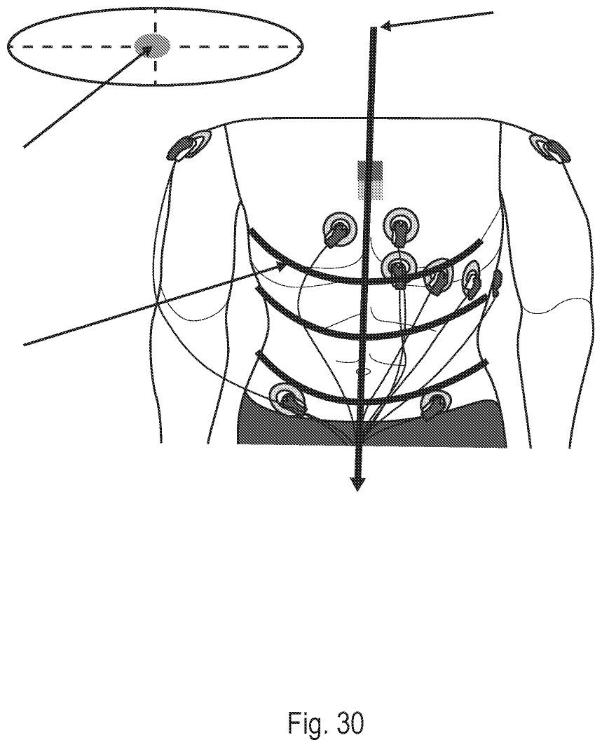

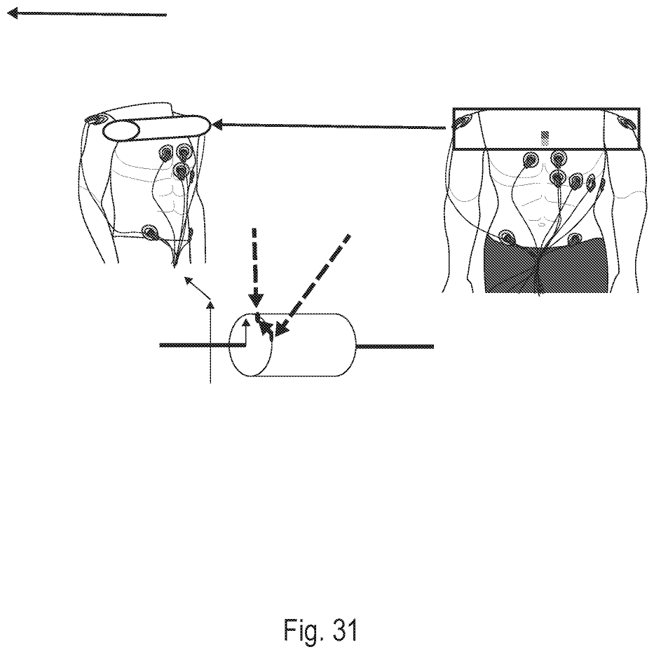

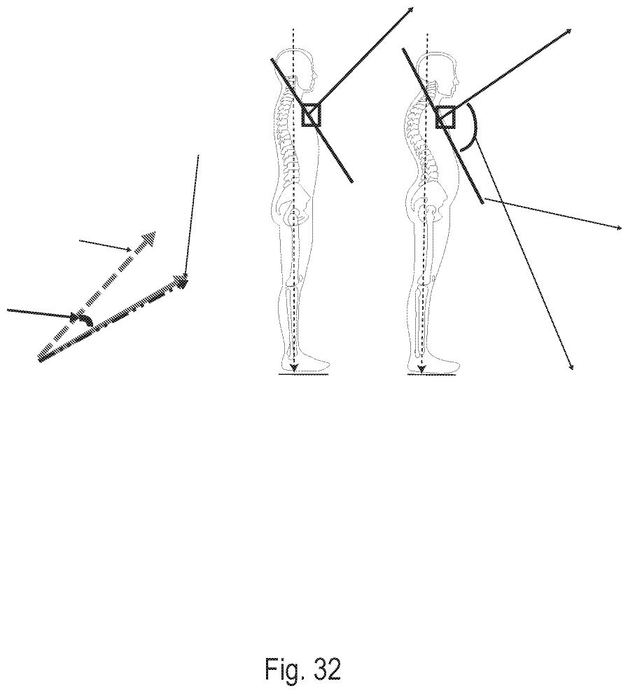

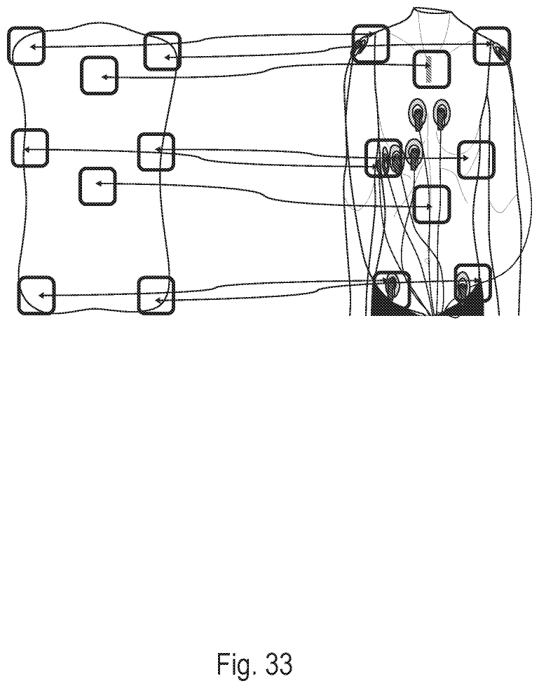

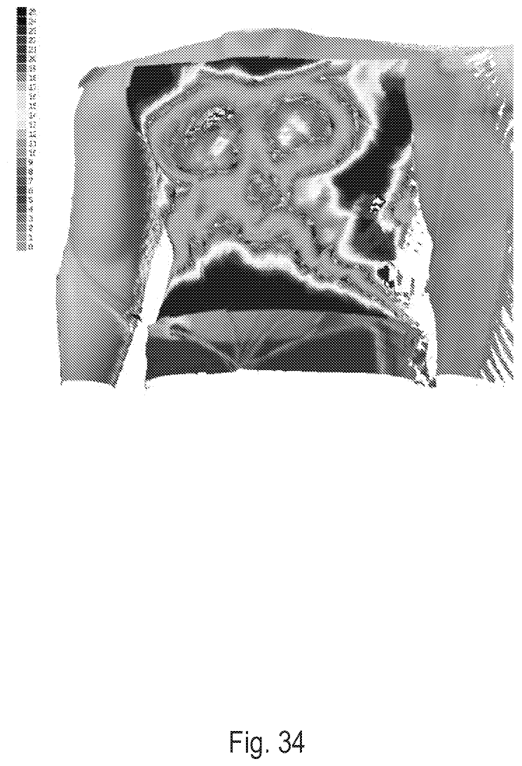

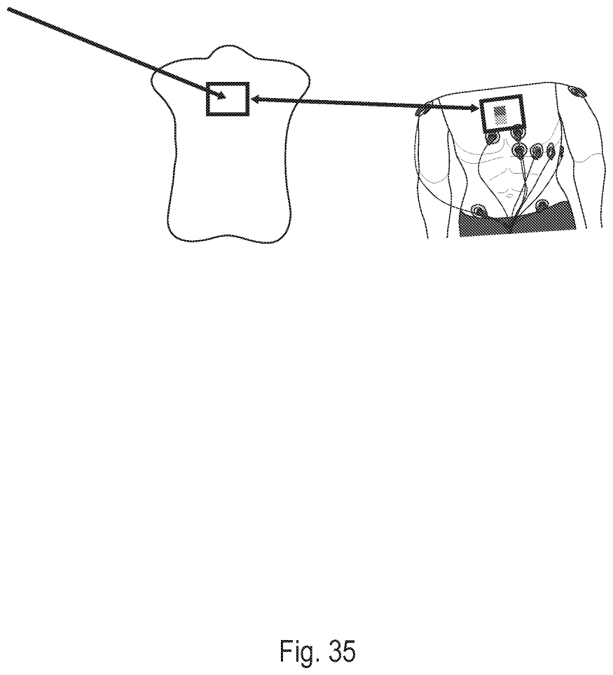

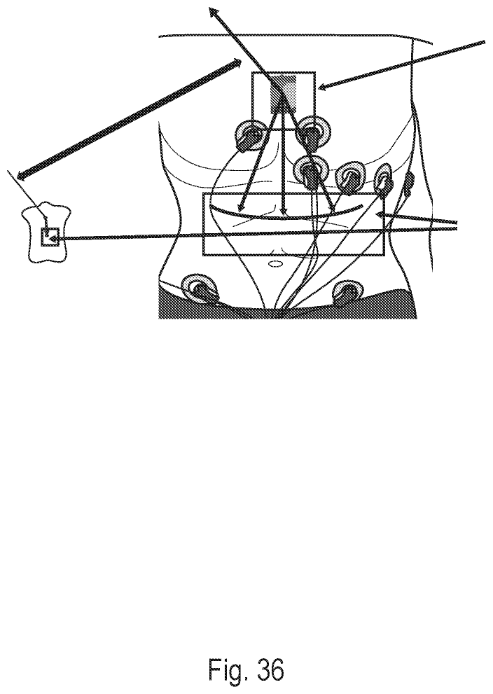

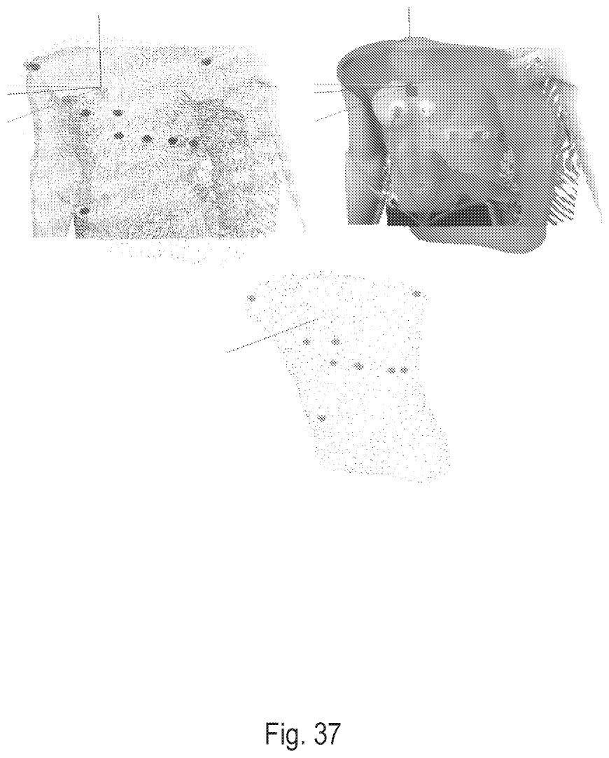

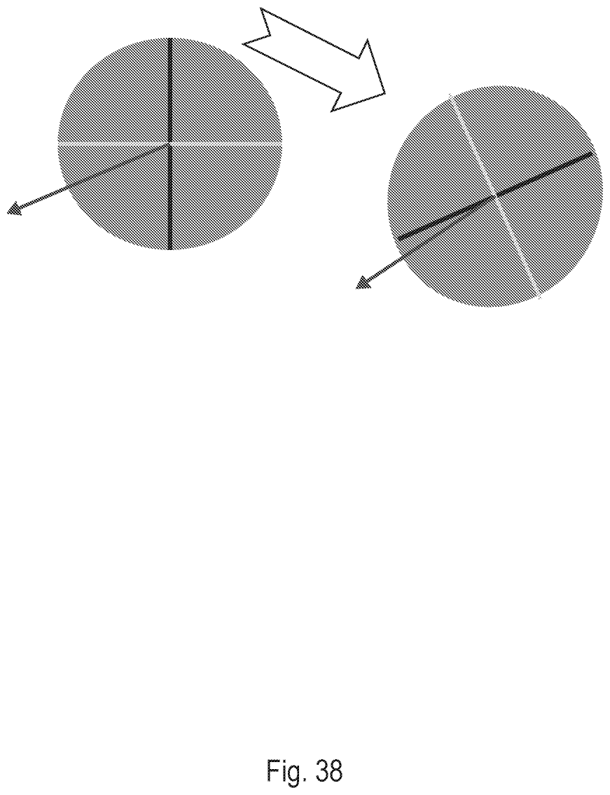

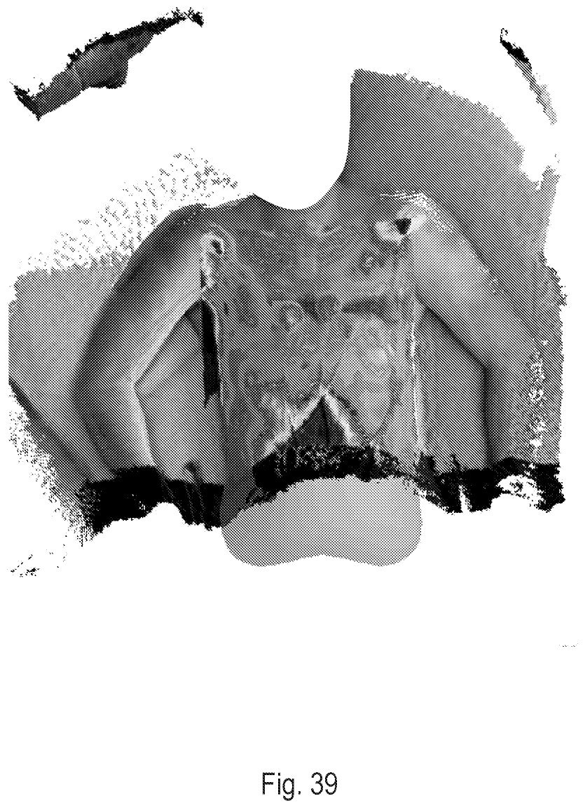

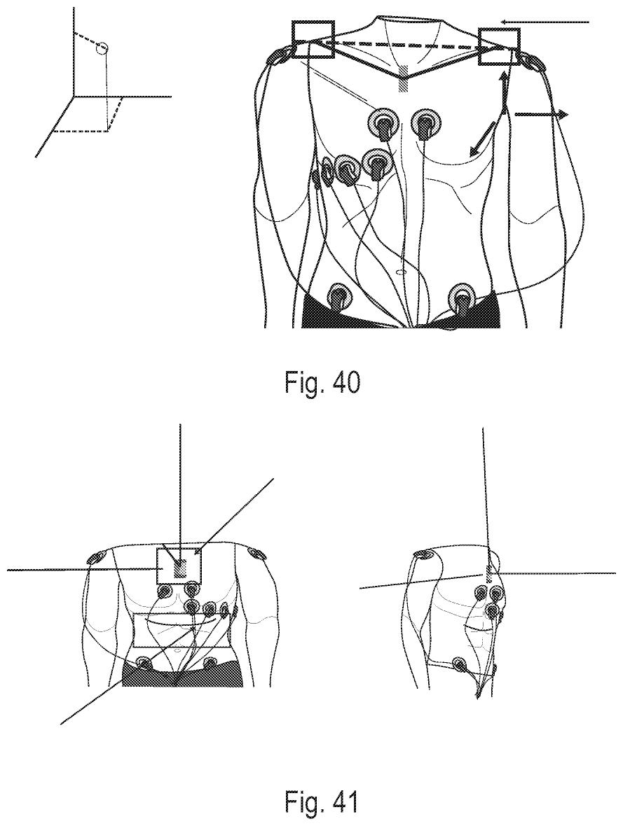

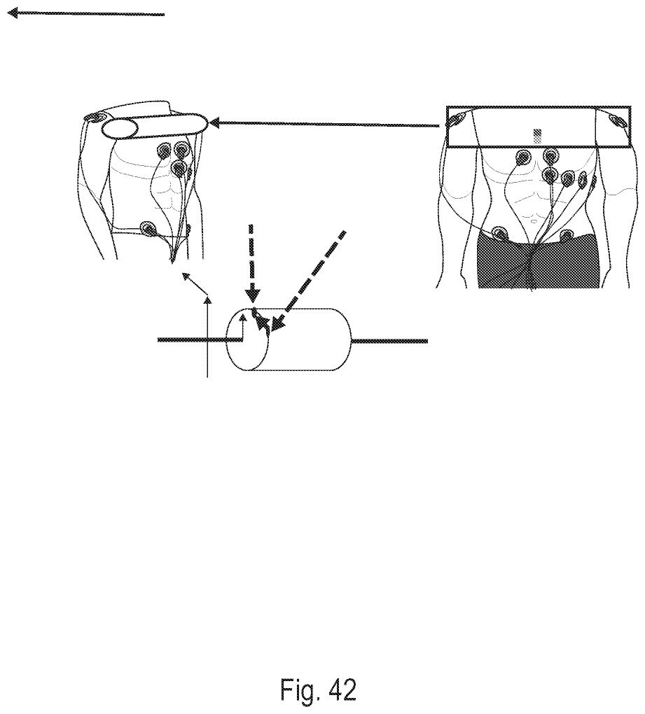

An advantage thereof is that such method allows for guiding an operator with attaching ECG electrodes at guided to locations on the torso. Furthermore, it allows to visualize or project location the operator is to attach the ECG electrode at the torso, or it allows to correct a the location of an ECG electrode that was momentarily placed at the torso, yet not at the correct spot. Again, the correct spot is a sport that provides a comparable ECG reading relative to an earlier ECG reading. This means that preferably the ECG electrodes are placed at the same spot as with the earlier ECG reading or as closely as possible also, it is preferable to define standardized ECG electrode placement spots or locations relative to fixed determinable locations on a torso. Preferably, such determination is made from the upper sternum and/or the lower sternum, further preferably in combination with a marker element, preferably according to FIG. 21.

Further preferably, such method comprises steps as described in the above according to the present invention and or embodiments.

According to a further preferred embodiment, the method comprises steps of visualizing of the feedback signal relative to the torso of the person comprises steps of at least one of the following:
visualizing the feedback signal relative to the torso in an augmented reality device;
visualizing the feedback signal relative to the torso by projecting the feedback signal on the torso by at least one projector, such as a video beamer;
visualizing the feedback signal relative to the torso by displaying the torso with a projection of the feedback signal on a display monitor;
visualizing the feedback signal relative to the torso by projecting the feedback signal on the torso by means of light spots from a laser projector. Such embodiment provides advantages relating to repeatability of placement of electrodes based on the placement of electrodes as in an earlier ECG measurement session. Because the locations of the electrodes of such an earlier session are indicated on the torso or a representation thereof on a display, repeatability of placement at the new or later session is easily achieved.

Further preferably, the location information comprises 3D location information as measured with at least one 3D camera. Further preferably, the method comprises steps of receiving patient information, such as a patient history comprising person characteristics relating to the at least one historical ECG measurement result and/or the historical ECG electrode location information.

According to a further preferred embodiment, the steps of obtaining historical ECG electrode location information comprises steps of obtaining historical torso shape information, preferably in which the steps of obtaining the latest or new ECG electrode location information comprises steps of obtaining latest or new torso shape information. Further preferably, steps are included for verifying that the historical data pertains to the person, such as by comparing differences between the historical torso shape and the latest or new torso shape.

Further preferably, the method comprises steps of assembling a graphical representation signal for displaying differences between at least one historical torso shape and the latest or new torso shape. Further preferably, the steps of receiving patient information comprises steps of requesting further patient information based on a patient identification, such as a person identification number, weight and/or height.

According to a further preferred embodiment, the method comprises steps of:
comparing historical ECG electrode location information of the at least one data point with the new ECG electrode location information,
rendering or assembling feedback information for providing a feedback signal as to an incorrect new ECG electrodes location, preferably with an indication of the correct location. Further preferably, the method comprises steps of obtaining latest on new ECG measurement results of the person with the location measured electrodes.

Further preferably, the steps of obtaining ECG electrode location information comprise steps of obtaining an optical, preferably 3D, recording of the torso of the person with latest or new ECG electrode locations of a placement position of the ECG electrodes. Further preferably, the method comprises steps of taking for differences or similarities between a respective historical torso shape and the latest or a new torso shape. A further aspect according to the present invention provides a system for application of a method according to one or more of the preceding claims, comprising:
a processing unit,
a memory coupled with the processing unit,
receiving means for receiving data entry identifying a person,
receiving means for receiving historical ECG electrode location information,
receiving means for receiving latest or new ECG electrode location information,
outputting means for outputting a feedback signal based on the historical ECG electrode information of the respective at least one data point.

Further preferably, said system comprises program coding means, and or processing means for performing any step according to the present invention.

Further advantages, features and details of the present invention will be described in the following in greater detail relating to one or preferred embodiments in the reference to the drawings. Similar yet not necessarily identical parts of different preferred embodiments may be indicated with the same reference numerals.

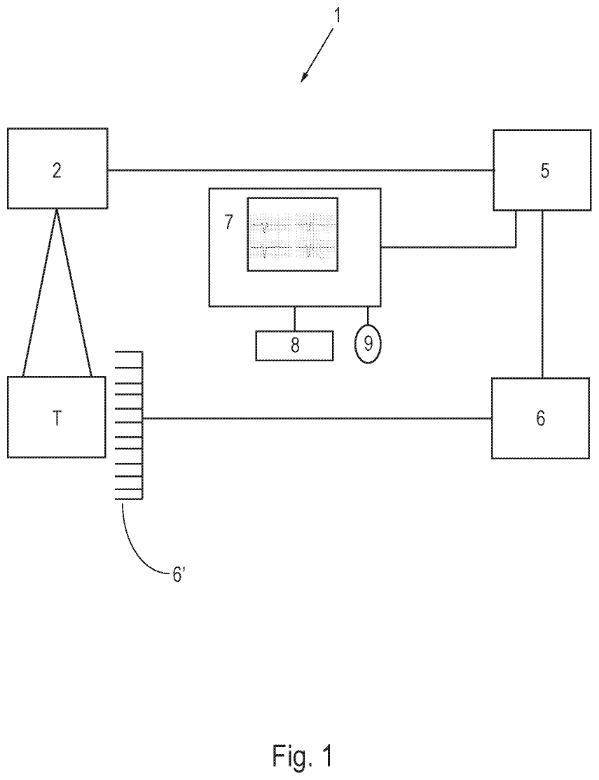

FIG. 1 shows a first preferred embodiment according to the present invention as an embodiment of a system according to the present invention for performing a method according to the present invention.

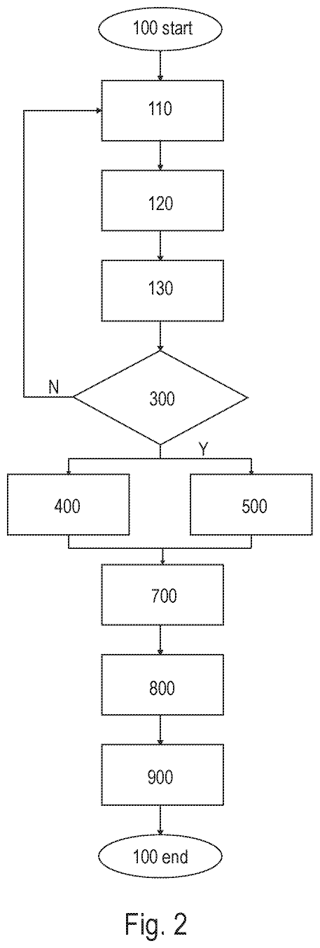

FIG. 2 shows a preferred embodiment of a method according to the present invention.

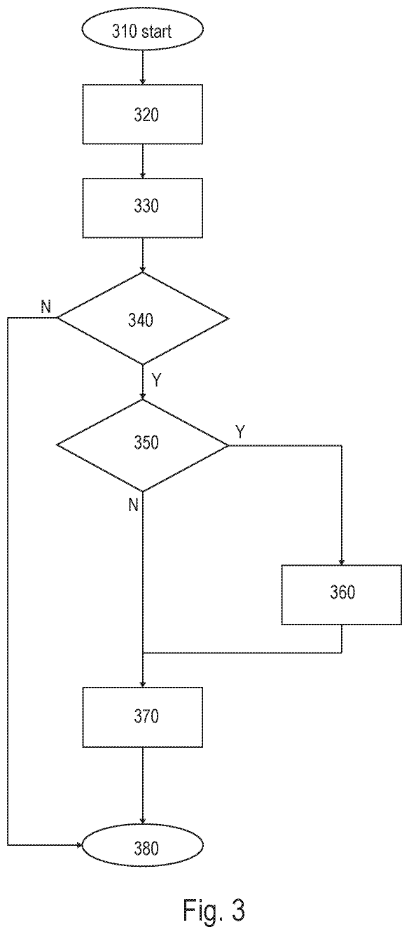

FIG. 3 shows a further preferred embodiment of the method according to the present invention.

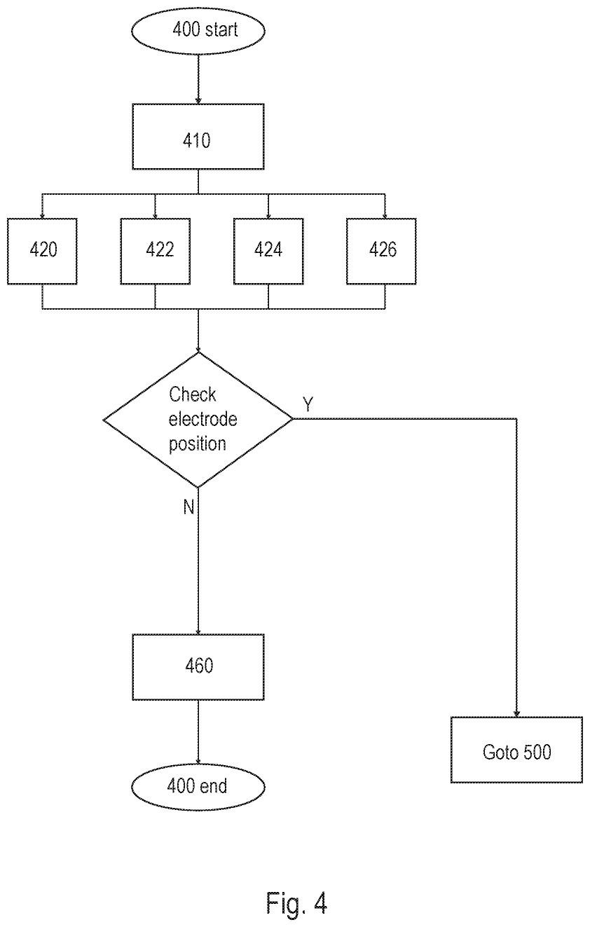

FIG. 4 shows a further preferred embodiment of a method according to the present invention.

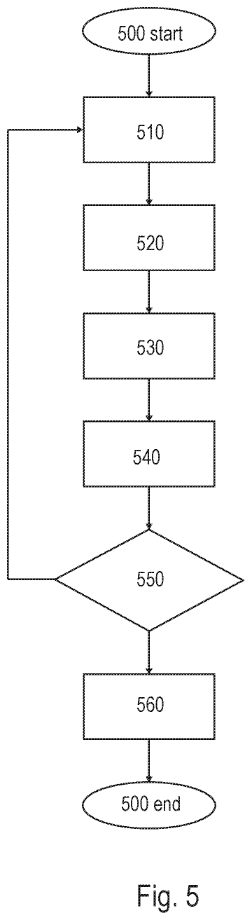

FIG. 5 shows a further preferred embodiment of a method according to the present invention.

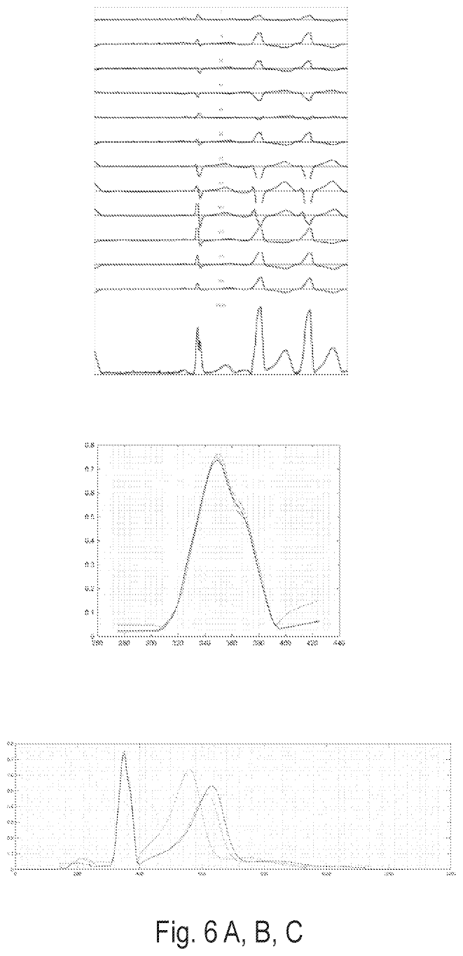

FIG. 6 shows graphical representations of an example presentable to a user on a display according to the present invention.

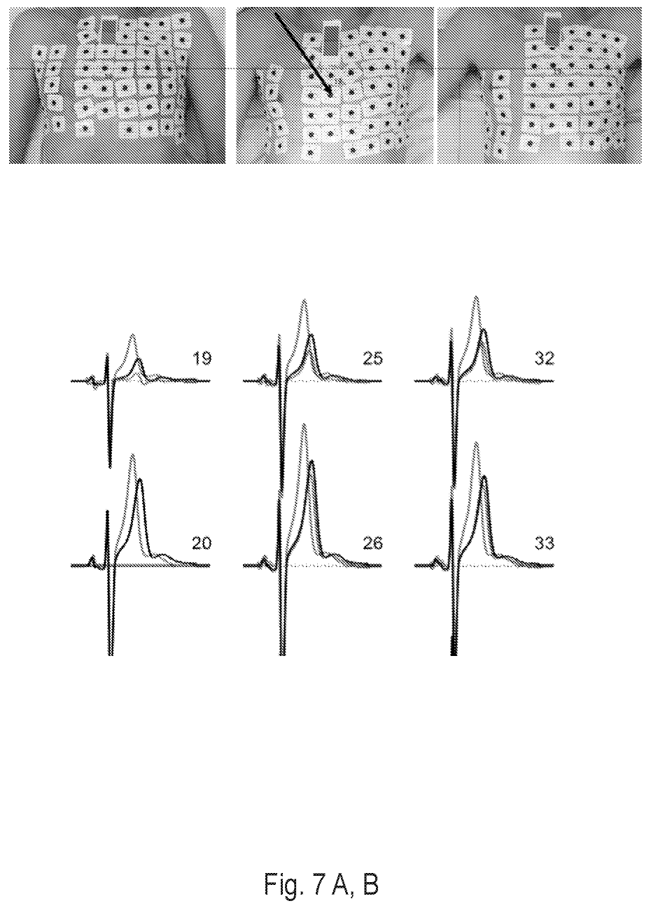

FIG. 7 shows an example of placement of ECG electrodes on a torso to gather with a marking element according to a further preferred embodiment of the present invention as well as graphical representations to be presentable to a user on a display according to the present invention.

FIG. 7 shows a further preferred example of presentable information to a user.

A first preferred embodiment according to the present invention comprises a system 1 for performing the method. It is envisioned by the present inventor at that the method may be performed during an ECG measurement session for a latest or new ECG measurement session. However, it is also envisioned by the present inventor that method may be performed after such session, for analyzing the new are latest ECG measurement session results with an earlier session relating to a respective data point.

A three-dimensional camera 2, for detecting ECG electrodes arranged at a torso T, is arranged above the torso T (schematically shown) of a person. The camera is suitable for moving thereof relative to the torso such that from several sides the torso can be recorded for detecting of the ECG electrodes in place. Data from the camera are transferred to a computer 5. The computer is connected to a monitor 7, keyboard 8 and mouse 9 for receiving input data from these peripherals from a user and for outputting of image data to the user. The computer is furthermore coupled with an ECG amplifier 6 that in its turn is coupled to ECG electrodes 3 on the torso T. A practical number of electrodes that is supplied is between 4 and 16, preferably substantially 12. A larger number for achieving a higher resolution is envisaged and use thereof dependent on the surroundings in which the installation is applied also usable. The skilled person would be able to determine the number of electrodes as a correct choice based on available equipment.

An embodiment for performing the method during an ECG measurement session, comprises the method according to FIG. 2. The method starts in step 100. In step 110, data entry identifying the person for obtaining historical data pertaining to at least one proceeding data point from a database is obtained. In step 120, data regarding to the person is obtained from a database, either a local database or a remote database accessible through a computer network.

In step 130, live 3D imaging information is obtained from a 3D camera 2. In step 140 optionally biometric values can be computed, more specifically facial recognition parameters, like distance between eyes, size of the head etc. In step 300, a check is performed with the purpose of comparing the live 3D imaging information, if present also the biometrics data, with the historical imaging information of a chosen data point. In case differences are too large to determine that the live 3D image pertains to the same person as from the historical imaging information of the chosen data point, the method returns in step 110 in order to obtain other person information. In case it is determined that the imaging information corresponds, the method continues in step 400 or step 500. Preferred details of step 300 are described with FIG. 3. Preferred details of step 400 are described with FIG. 4. Preferred details of step 500 are described with FIG. 5.

In step 400, it is checked whether the ECG electrodes of the live session correspond sufficiently with the information from the historical data point. Location information as to the historical data point is projected on the torso of the person in order to place the electrodes of the live ECG measuring session at the exact spots as projected. An alternative of step 500 is that the ECG electrodes of the live ECG measuring session were already placed and a relevant projector projects indications as to incorrect placement.

In step 700, the ECG recording of the live ECG recording session is made in order to obtain the latest or new ECG measurement results of the person with the location measured electrodes. In step 800, the user interface is displayed with the waveforms of a chosen historical data point and the live, new or latest ECG measuring session. The method ends in step 900.

In step 320, the live 3D recording of the thorax is matched with respective or chosen historical data points from the database in order to determine whether the thorax corresponds sufficiently. In step 330, a match error is determined as disclosed below. In step 340 it is determined whether the error is below a predetermined threshold, in which case the method continues in step 400 or 500 according to FIG. 2. In case it is determined that the error is above a predetermined threshold, it is determined in steps 350 whether the shape or build of the torso has changed. In case the torso has changed 360, the torso model based on the 3D imaging information is adapted in order to be able to continue. In 370 an extra check on the identity of the person of the torso is performed using biometrical parameter values from the 3D photo, more specifically facial recognition. In 375 these parameters searched in in the database and when found with a match the identity is confirmed. In case the patient is not confirmed with the database, the method proceeds in step 380 storing the torso model or updated torso model.

In step 410, a production method is chosen based on availability of a relevant subsystem or a preference of the person. In step 420, an augmented reality projection is used in order to projected the electrode locations on the torso in a way that the person placing the live ECG electrodes sees a projection on the torso. In step 422, a beamer is used for this purpose. Such beamer is capable of projecting spots and/or additional information as to the correct electrode to be placed on such a spot. In step 424, the torso is projected on a display monitor together with information spots a relating to the live markings as placed on the torso and the correctness thereof. In step 426, a projector projecting the laser beams is used for projecting spots and or additional information as to the correct location of the ECG electrodes on the torso.

It is either provided that one projecting device is used that is moved during the procedure or that 2 or more projecting devices are used for projecting all spots or locations on the torso simultaneously. Similarly, one, 2 or more 3D imaging devices are envisioned in order to make the recording of all spots simultaneously or by moving one such camera.

In step 510, the electrode positions are determined from the 3D recording of the live ECG session using their 3D thorax model as indicated below in the description. In step 520, the electrode positions of the live recording are compared with the electrode positions from the database from the determined data point used. A difference thereof is determined.

FIG. 6 A shows an example of the ECG signals. FIG. 6B shows a detailed QRS fuel on 3 different RMS signals of the 67 ECG signals in the BSM. FIG. 6C shows the aligned P wave, QRS complex and T wave, the QRS is the same. The T wave of the second measurement is different, because the subject had a higher heart rate, consequently the T wave (recovery of the heart) occurs earlier.

FIG. 7 shows the placement of the ECG electrodes relative to a marking line. This clearly shows how the electrodes are sometimes placed higher than other times. The deviations of such placements are shown in the graph of FIG. 7B.

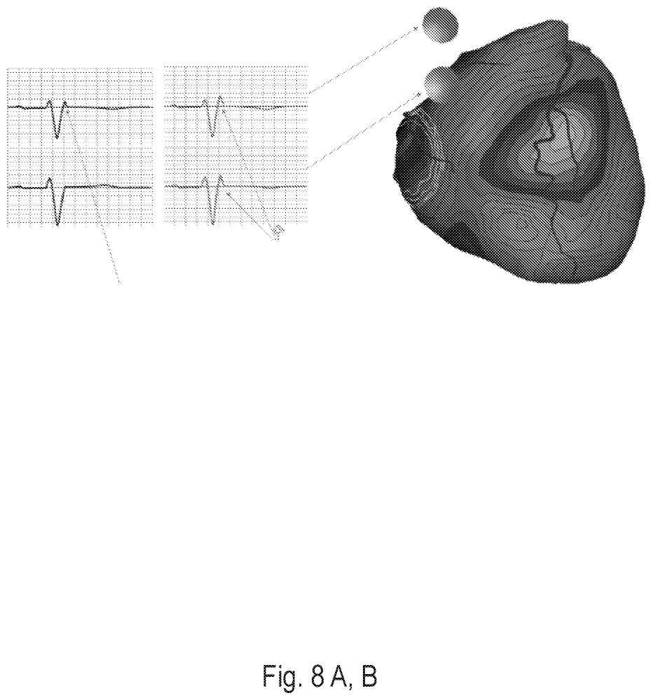

In FIG. 8, the initial stage of an ARVC is shown in the heart model by the darker left side. It is shown in the graphs how the ECG signal of the electrode fee one placed too high relative to the heart on the torso leads to a similar wave pattern as the wave pattern of the correct placement below that shows a wave pattern belonging to a condition. Because of this deviation due to an incorrect placement, the initial stage of the ARVC could not be recognized if it was not made sure according to the present invention to place the electrode at the correct position.

As indicated in relation to an above preferred embodiment, the use of the projector provides advantageously feedback with regards to where electrodes are to be placed in alignment with a respective earlier ECG recording, and/or a deviation of a placed electrode with regards to a respective placement during such respective earlier ECG recording.

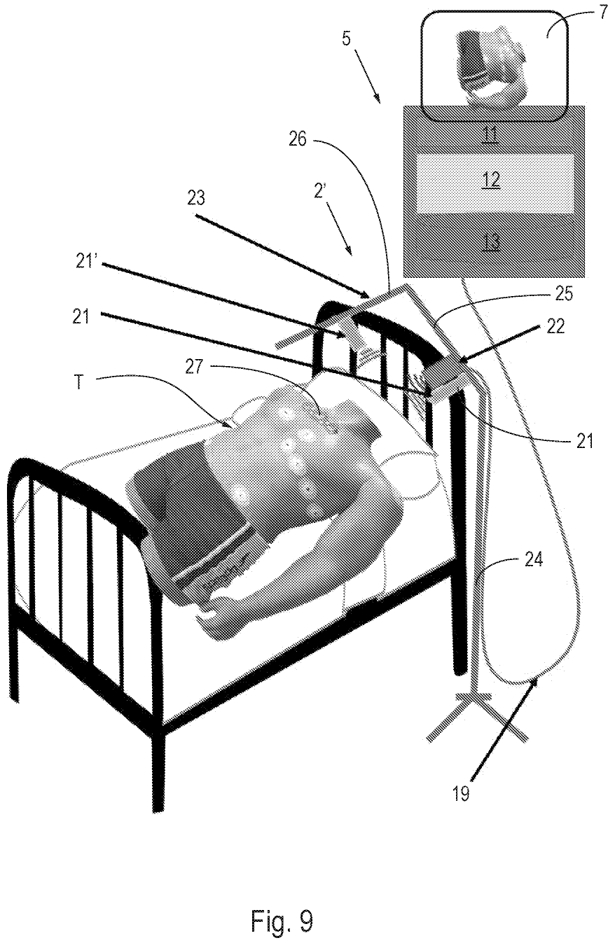

To this end, according to the preferred embodiment of FIG. 9, a camera projector unit 2′ is provided, which is being connected to a computing device 5 comprising a user interface, the computing device 5 comprising functional elements to provide a user interface 11, functional elements to receive information from the camera and to send instructions to the projector 12, and a storage medium for storing data, retrieving historical data and retrieving data with respect to the patient.

A stand 24 comprising extending arms 25, 26 are arranged to be extended over a patient with a torso T. In this example, the projector 22 is combined with two cameras 21, 21′. In other embodiments, two projectors are combined with two cameras or two projectors are combined with one camera, depending on functional requirements. Preferably, a 3D camera is combined with a projector in a housing. Such a projector with a housing is preferably arranged above a patient, further preferably two 3D cameras with two respective projectors are arranged at either arm of an embodiment of a stand as shown. Preferably, such arrangement is such that the torso can be recorded by the cameras and projected ads by the projectors from generally two sides in order to cover both the top of the torso and theater sites. Especially the left side is preferably reached as that is the side of the heart at which side more ECG electrodes are to be located. Also a marker elements 27 is arranged at the torso, preferably at the top site thereof, preferably at the upper part of the sternum or suprasternal notch and having the marker element positioned along the sternum. Also a marker element at the bottom end of the sternum is envisioned as well as a combination of two of such marker elements. As a 3D camera, also the inclusion of a time-of-flight camera is envisioned for providing 3D or depth information for both the camera recording and projection purposes.

A unit comprising a camera and a projector preferably comprises also a computer unit with control functions for the camera and the projector and with communication functions for communication with the computer 5 of the preferred system. Preferably, the projector and 3D camera are contained inside a housing attachable to the stand 23 to be positioned relative to a person of rich ECG recordings are to be taken. Insight of the housing, means for adjusting projections are arranged, such as 2 or 3 axis, perpendicular to each other relative to which an internal frame is movably attached. Relating stepping motors are arranged suitably to move the parts relative to each other. With this, it is achieved that changes in the position of the camera and of the projector relative to the torso are adjusted for in order to provide suitable recording and suitable projecting relative to the patient. As such, the projector is suitably alignable based on interpreted image recordings by the 3D camera, relative to the person of each the ECG recording is taken. This allows for moving the whole projecting area relative to the torso.

The projections as projected by the projector are recordable by the camera. Such recorded projections are compared with intended points of projection in order to determine or verify the correctness of the projection based on this determination, the computing device calculates corrections based on which the projections are adapted to be improved. In case of incorrect projections, the operator is notified thereof by means of a suitable warning signal, such as a sound, and adaptation of the projection, and interruption of the projection, such as intermittent projection or a changed coloration of the projection.

Envisioned embodiments of the projector comprise an LED projector, a laser projector, a laser pointer and/or a micro electromechanical system with laser point scanning, or the like. The purpose of the projection is to provide a clear indication of ECG electrode locations. As such, embodiments merely providing point or line projections are preferred, although even laser pointer based micro electromechanical systems would also be capable of providing graphical pictures that are envisioned in preferred embodiments for providing supporting imagery. It is preferred to use the 12 lead ECG recording need configuration, preferably with a main focus on 6 frontal chest electrodes on the torso. All ECG lead configurations are envisioned supportable with a suitably embodied embodiments within the understanding of skilled person with the disclosure of this document.

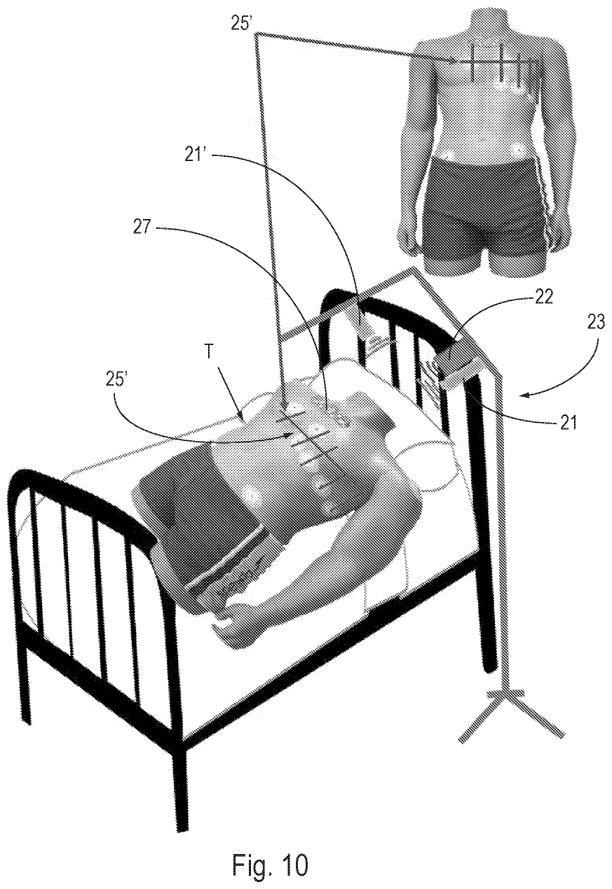

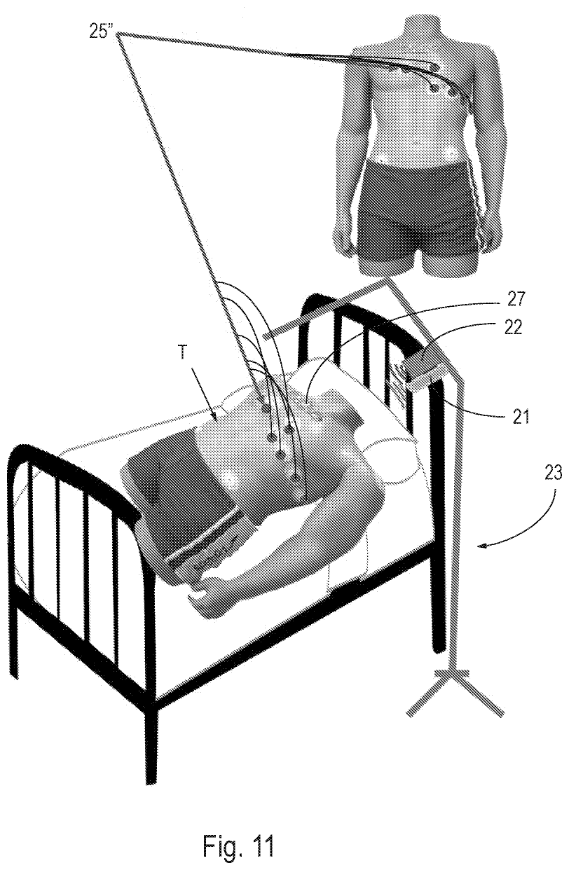

FIG. 10 generally shows the arrangement with cameras and projector according to FIG. 9. In FIG. 10, an example of a projection is shown, which example indicates a number of crossing lines, the endings of which indicate a location of a respective electrodes to be placed at the torso. The lower lying down torso indicates an example in which electrodes are placed in a general pattern by an operator as the operator is used to during normal ECG recording operations, such as in normal preparation of a person. However, the lines indicate positions that deviate from the positions at which the ECG electrodes are located. These ECG electrodes that are to be moved to the desired locations in order to achieve the desired result of this ECG recording in a line of ECG recordings over time. Such a result is not indicated that the normal preparation is wrong, but it indicates how the invention provides an improvement for the desired results to be achieved. There vertically arranged torso of FIG. 10 indicates the ECG electrodes or parts thereof. A camera detectable marker element 27 provides input to the computing device 5 for performing operations according to embodiments of a method according to the present invention in order to make determinations as to the shape of the torso, placements of electrodes thereon, such as in line with earlier ECG recording sessions.

Alternatively, FIG. 11 shows projection points 25′ to be projected onto a torso at locations ECG electrodes are to be positions. Also in this Fig., a marker element is arranged at the upper part of the sternum helping with determining the position of the electric positions on the torso, by means of image recordings by the camera.

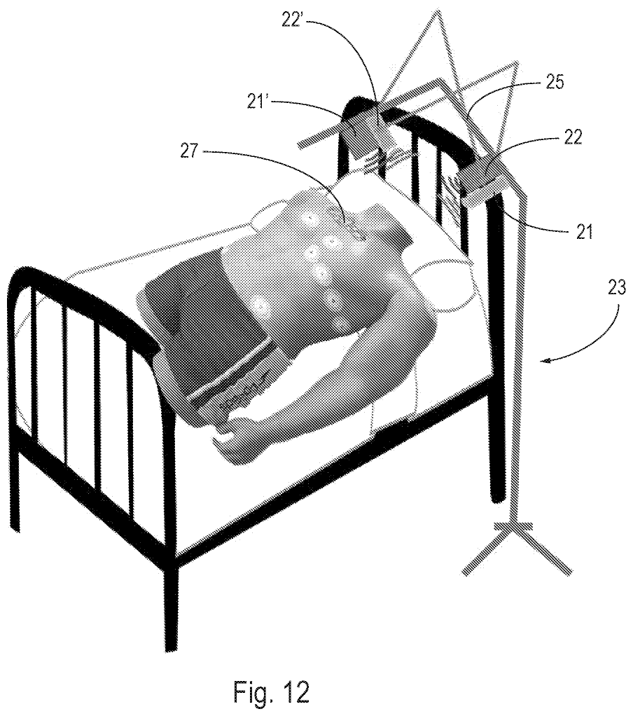

FIG. 12 shows a similar arrangement in which 2 cameras and 2 projectors are arranged at the stand 23, one camera projector combination at arm 25 and 1 camera projector combination at arm 26. A preferred angle between the arms is about between 90 and 150°, preferably between 100 and 140°, more preferably between 110 and 130°, more preferably about 120°. In order to emphasize the left side of the torso, and overall tilt to that left side from straight above is envisioned.

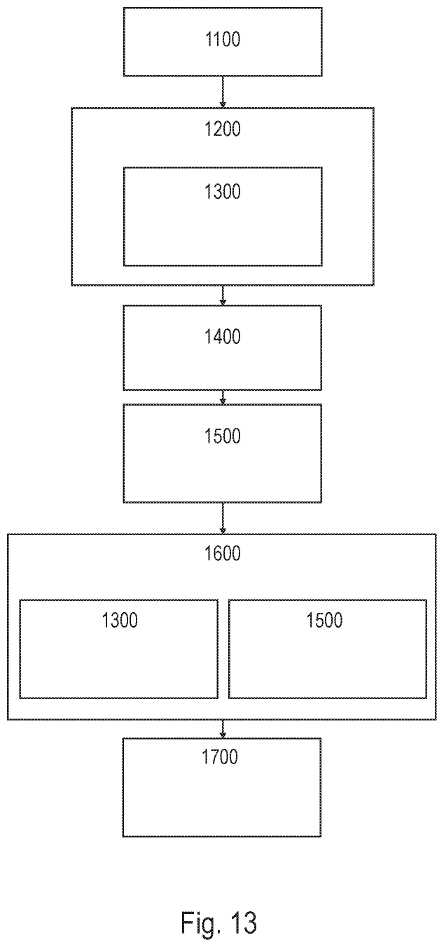

FIG. 13 provide the depiction of a main workflow of a further preferred embodiment according to the present invention. The method starts in step 1100 with an initialization of the system, a loading of a configuration of attached components such as one or more 3D cameras, one or more projectors, stepping motors, recording information relating to a person regarding earlier ECG recordings, and or respective lead configurations, such as comprising locations on the torso of respective ECG leads of such early recordings, and preferably performing initialization of imaging. In step 1200, the relevance devices as determined in step 1100 are started and initialized with configuration settings like frame rate and recording image resolution. Furthermore, the initial position of a camera relative to the patient is checked, and adjusted or indicated to be adjusted to an operator. In step 1300, of each step 1100 detected projector, the projector is started, initialized with configured settings and calibrated with use of a calibration image and/or calibrated relative to the camera.

In step 1400, torso characteristics are obtained from 3D camera recordings. In step 1500, relevant information from previous steps are processed for preparing the projection of the ECG lead location on the torso. Further preferably, patient characteristics derived coordinates, such as from step 1300, loaded previous recorded lead position coordinates and or a default position rest or, such as from step 1100, are applied.

In step 1600, relevant ECG lead locations are projected with the relevant projector onto the torso and in interaction with an operator is provided for attaching the ECG electrodes at the torso. Further preferably, checks are provided if the operator attached the leads at the correct position such as the locations of the previous recording or indicated default locations adjusted to the relevant torso. In case ECG electrodes are placed incorrectly, this is recorded with camera imagery and the operator is instructed by means of relevantly provided projections and/or a user interface on the computer to adjust the relevant ECG electrode or electrodes. In step 700, all relevant retrieved and write information from the present ECG session and electrode placement locations are stored for later use or analysis.

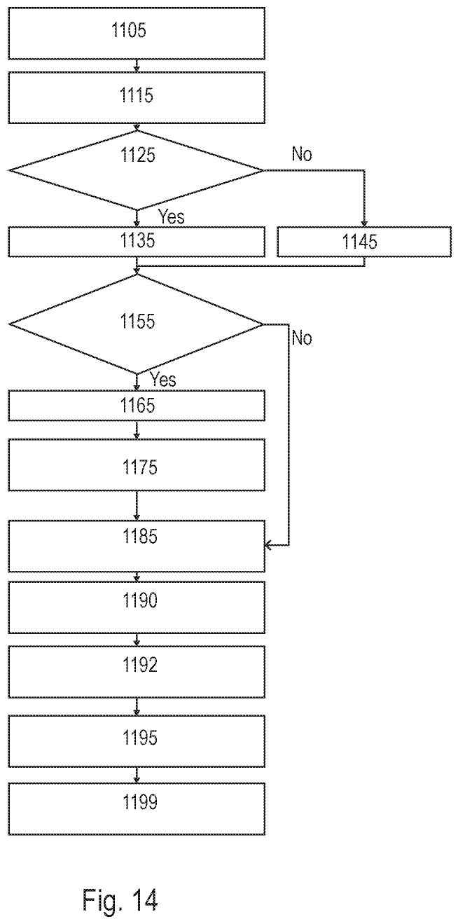

FIG. 14 provides preferred steps detailing step 1100 of FIG. 13 with optional steps or substeps. In step 1105, the number of attached 3D cameras attached to the system is determined or retrieved. This information is either retrieved from a local storage or retrieved by means of communication with attached cameras such as with a query. In step 1115, the number of projectors that are attached to the system is retrieved. This information is either retrieved from a local storage or retrieved by means of communication with attached projectors, such as with a query. In step 1125, checks or requests are performed, such as from or through the user interface or storage medium, if previous recordings of ECG lead locations such as with previously stored coordinates and recording information are available for a torso of which in the ECG recording is to be made. If such data is available, the respective reference points and locations of the ECG electrodes are obtained. Preferably, a previously created torso model is loaded, if such previously created torso model based on 3D imaging such as CT or MRI is available. With such 3D torso model, optimizations with respect to the ECG electrode locations are performed.

In step 1135, the previous recordings of ECG electrode locations with relevant previously stored coordinates and recording information are retrieved and stored in operational memory. This information is either retrieved from a local storage on the local system or from a connected storage medium. Search loaded previous recording configuration preferably includes previously determined torso model, optionally a position of a marker element, reference points of the torso, earlier determent torso characteristics and/or locations, relative to the reference points and/or marker, of the ECG leads during relevant previous recordings.

In step 1145, preferably default ECG lead locations are retrieved or set of projection instructions with coordinates and production information thereof. Such information is either retrievable from a local storage on the device or from a connected storage medium. Such information will contain reference points to be determent in the 3D recordings like top side and bottom side of the sternum, shoulder positions, up/down/left/right/top/bottom orientation and locations, relative to reference points/marker element, of the ECG electrode configuration. This information is subsequently stored in memory. In step 1155, it is determent what markers are to be used during the current recording session. This information is for example retrievable from a from a storage or provided by the operator via the user interface. The marker element is optional as individual body parts may be recognized from camera imagery to preferably base location determination of the torso in the recording information of the camera imagery, or in case a default raster is being used. Body parts to be used for such determinations comprise arms, head, nipples, breasts or chosen reference points, such as pointed out in the camera imagery or derived from previous recordings.

In step 1165, optionally marker information and characteristics to find in the camera imagery are retrieved from storage or operator input. Since characteristics of the marker element comprise for example geometrical shapes, colors, geometrical position shapes. Further information regarding to the marker is described below. Marker is positioned on the torso at a defined position, such as the top of determine, and in a defined orientation. This information is stored in memory to be used in subsequent steps. In step 1175, the operator is instructed by means of the user interface how and where to attach the marker element to the torso. In step 1185, calibration images to use our retrieved. Such information is either available at a local storage or at a connected storage medium. A connected storage medium, in any embodiment, in the context of this disclosure entails both local disks or remote databases. Such calibration image is a predefined and known image or a set of projection instructions comprising or forming a raster and or geometrical shapes with defined calibration coordinates relative to torso characteristics.

In step 1190, and ECG electrode configuration to be used is loaded. This information is either retrieved from a local storage or a connected storage medium. A standard 12 electrode configuration is preferred, but all known ECG electrode configurations may be applied with suitable adjustments to the embodiments within the scope of a skilled person having the disclosure of this document. In step 1192, a configuration of the type of ECG electrodes or patches and connectors to be used are loaded. Such information can either be retrieved from a local storage on or a connected storage. This information comprises shape and color information, such as a round white circular shape with a white elevated circular center with metal electrode connection nipple and rectangular connector with different color per ECG electrode, or any known ECG electrode type information. In step 1199, the relevant information to perform the method according to preferred embodiments is loaded in memory of the computing device to be ready to be used. Such information comprises torso characteristics, calibration image, previously stored recording data or a default raster, whether a marker element will be used, what type of ECG electrode will be used, and what ECG electrode configuration will be used.

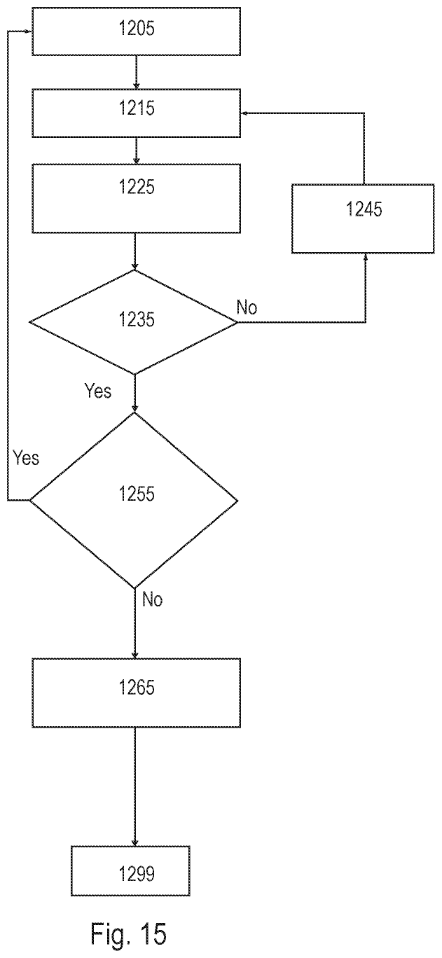

FIG. 15 discloses preferred steps of step 1200 of FIGS. 13. In step 1205, relevant camera information such as frame rate, recording image resolution and relevant 3D camera. The interaction with the 3D camera is subsequently started. In step 1215, an image is recorded with the 3D camera or the 3D cameras. In step 1225, a marker element is located in the recorded image based on characteristics thereof and or body parts with derived coordinates configured to be recognized based on such characteristics. To this end, step 300 is used as part of step 1225. The marker preferably supports readily identifying of the torso. To this end, the curvature found within the 3D image and colors in combination with the resulting shape of these comment areas are used to be matched to predefined criteria. Such criteria are the symmetry of the torso, shoulders in combination with for instance the neck and the face. In step 1235, and identification of the marker element and or the torso for tracking thereof in the images is checked. The torso is to be correctly identified and sufficient line of sight towards the area in which the ECG electrodes are to be located based on the ECG electrode configuration of step 1190 is to be determined. In step 1245, the operator is instructed by means of the user interface in case adjustments with respect to the stand 23 and/or an arm of the stand are to be made for correction of the orientation of the device relative to the torso. The user interface is optionally used for providing instructions to the operator in performing such correction by indicating relevance directions in which to move the stand and/or the arm.

In step 1255, if optional further cameras are configured/found, step 1205 is performed relative to such device. In step 1265, in case multiple 3D cameras are used, and alignment transformation between the images based on for instance the optional marker and or body characteristics determined in steps 1225 through 1395 are determined for the images taken from the relevant cameras. Transformation of each relevant 3D image derived party characteristic coordinates are determined by adjusting such coordinates until an overlap with their first 3D image is achieved, such as by translating and/or rotating the relevant device. In step 1299, all cameras are activated, inspection is operational and the stand with the arm are correctly positioned relative to the torso. And alignment transformation aligning 3D images from multiple cameras is stored into memory.

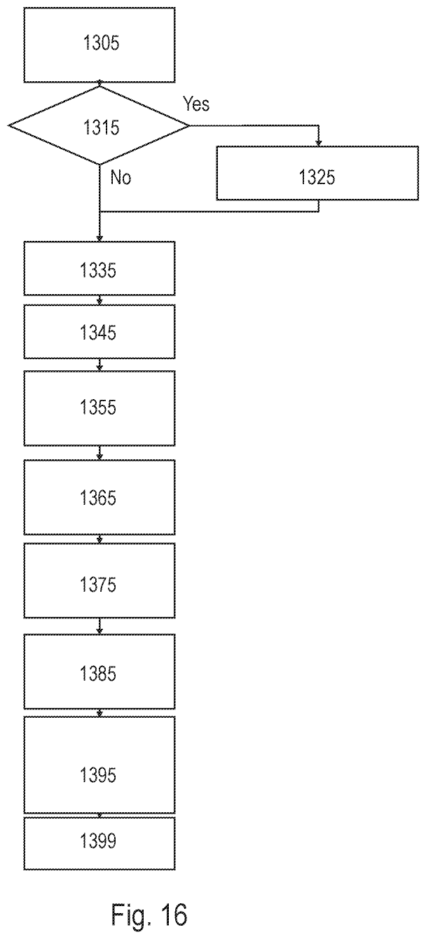

In FIGS. 16, preferred step 12 step 1300 are disclosed. In step 1305, an image recorded in step 1215 is loaded or retrieved. In step 1315, it is determined by Dr. marker element is configured to be used. In step 1325, the marker element is located in the images based on marker characteristics retrieved in step 1165. The marker is used as a known element in the image and as such as reference point relative to reference points to be tracked in the images, such as ECG electrode locations.

In step 1335, shoulders are located in the image based on body characteristics retrieved in step 1195. Information, such as potential shape, curvature and expect the distance between the left and right shoulder, preferably with a neck in the middle is used as reference information. If a marker element is present in the imagery, shoulders are located within a defined area relative to the marker elements. Based on such shoulder information, a solar line is estimated, which defines a border line at the top of the torso. Such top of the torso is also used for and minimization of identifiable information like to face upwards of such line. Such line is also being used in the next step.

In step 1345, based on body characteristics retrieved in step 1195, sides of the torso are determined in the imagery. This is preferably performed based on a line over the middle of the shoulder to shoulder line and substantially being perpendicular to that shoulder to shoulder line as derived in the previous step. From this middle chest line, curvature is to work the left and right sides of the torso are being determined. If a marker element is present in the imagery, the line over the middle of the chest may be determined within a defined area and direction relative to the marker elements. In step 1355, the just circumference is determined from the imagery, preferably based on the body characteristics retrieved in step 1195 and/or a formation of elliptic circles derived from such curvatures. In step 1365, preferably based on torso characteristics of step 1195, the location of the shoulders as determined in step 1335, the just circumference determined in step 1355 and/or the marker element location determined in step 1325, the length and location of the sternum is determined from the imagery. Optionally, the operator provides information include for validating the determined length and location of the sternum. In step 1375, the presence of breasts is optionally determined based on for example torso characteristic information retrieved in step 1195.

In step 1385, preferably based on information of steps 1335, 1345, 1355, 1365 and/or 1375, initial 3D images are taken or adjusted based on subsequent images to provide a new model of the torso.

In step 1395, based on information retrieved from the steps 1335, 3045, 1355, 1365, 1375, 1385 and/or step 1325, preferably in conjunction with torso characteristics from step 1195, torso information and coordinates to track are extracted. Such are preferably the top and bottom of the sternum, the sides of the torso and the line defined by the shoulders and further optional reference points preferably used to project ultimate ECG electrode locations. Such will be tracking coordinates. In step 1399, torso part tracking coordinates with torso characteristics and preferably a new patient model will be stored to memory.

In step 1405, first or further projects or information like projecting resolution and available projectors are retrieved. Interaction with the projector is started. In step 1415, the projector is instructed to protect the calibration image. In step 1425, a recording from relevant 3D cameras is obtained. In step 1435, calibration images are obtained from the recorded 3D image, such as based on color and shape and calibration coordinates, such as according to step 1185 are determined there from. A configured relation of coordinates with the torso characteristics and tracking coordinates tear off I determined. In step 1445, differences between derived coordinates of sets calibration coordinates of the calibration image and expected coordinates are determined relative to torso characteristics and found tracking coordinates. In step 1455, adjustments are made in case projection sharpness is insufficient. In step 1465, based on differences in coordinates determined in step 1455, a correction of the projector frame is performed. In step 1475, in case coordinates determined in step 1445 indicate a deviation, a correction of size, width and/or hide of the information to project is performed. In step 1485, the presence of any further projectors is checked. If such further projector is present, step 405 is re-performed for such projector. In step 1499, it is determined that all projectors are activated, interaction is set up and calibration has been performed relative to the torso and the 3D cameras.

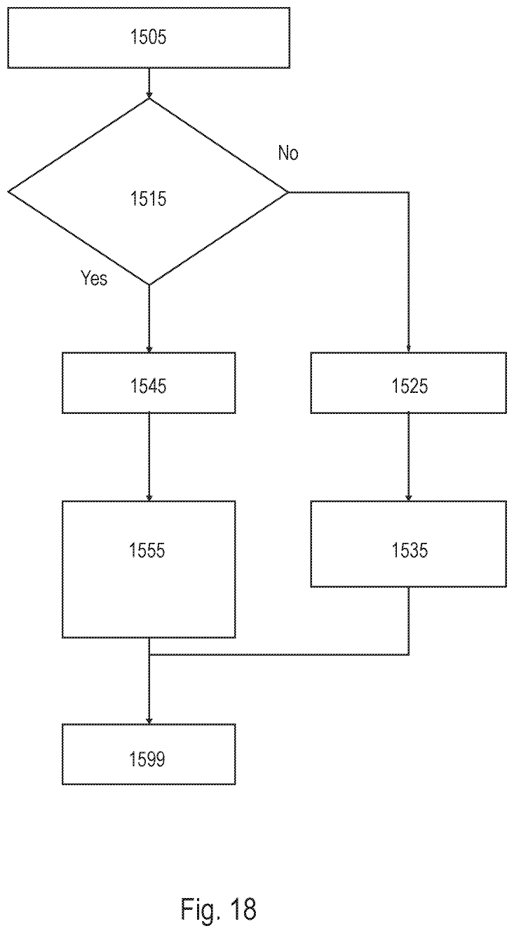

In step 1505 (FIG. 18), torso tracking coordinates are loaded. In step 1515, it is checked if a previous recording is available. In step 1525, in case no previous recording is available, default ECG electrode locations are determined to be used for projection. In step 1535, coordinates of default ECG electrode locations are adjusted to fit the determined body parts coordinates and/or the torso. In step 1545, it is determined that a previous recording is available. Body parts tracking information is loaded from that previous recording together with the retrieved ECG electrode locations relative to that. Distinct offsets for such previous ECG electrode locations relative to a reference coordinates from that recording are determined.

In step 1555, coordinates of the offsets are adjusted to the ECG electrode locations of the previous recording to the equivalent coordinate in the current determined torso tracking coordinates. This provides projection points which are applied as target electrode coordinates to memory. In step 1599, the image to project the ECG electrode locations on the torso has been prepared and the target electrode coordinates are saved to memory.

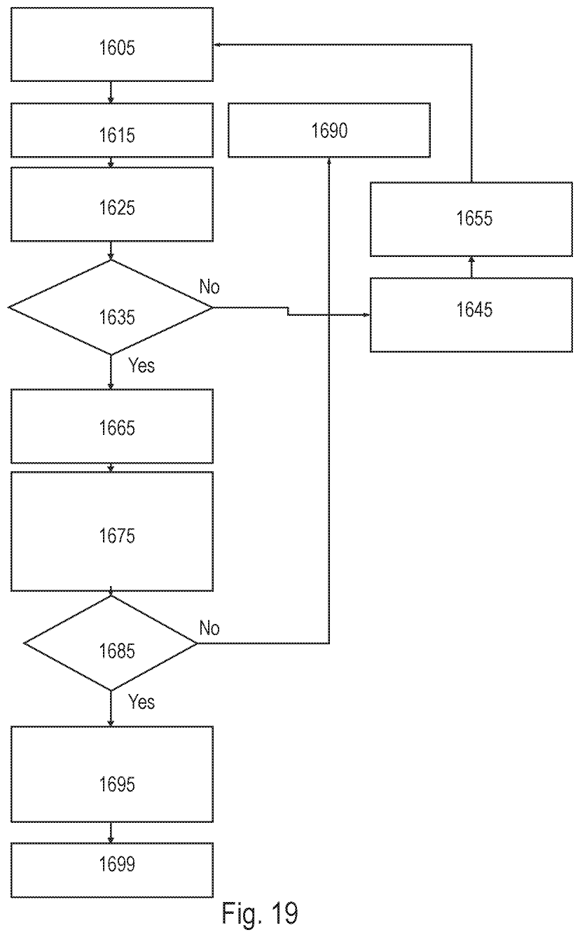

In step 1605 (FIG. 19), the image to project the ECG electrode locations are projected on the torso. In step 1615, graphic user interface provides instructions with regard to placing electrodes or correcting placing of electrodes. In step 1625, and image is retrieved from the relevant 3D camera. In step 1635, it is determined whether tracking coordinates correspond with corresponding torso characteristics in the image based on color and shape information at the tracking coordinates and a surrounding area thereof. In case a deviation of more than for instance 2 mm is detected, the method proceeds in step 1645.

In step 1645, tracking coordinates and torso characteristics are calibrated by re-performing step 300. In step 655, the image to project is updated based on changed tracking coordinates and torso characteristics by reperforming step 500. In step 1665, the ECG electrodes are located in the recorded image based on the configuration of the type of ECG electrode and connector. This is performed based on provided information with respect to shape, color and height of such ECG electrodes. In step 1675, the found coordinates from the previous step are compared with target ECG electrode coordinates from step 500. In step 1685, it is checked if all leads are correctly located. In step 1690, if one or more leads are not correctly located, the the user interface will provide a feedback signal and return to step 1615. In step 1695, all X will ECG electrode coordinates are stored to memory. In step 1699, final tracking coordinates and torso characteristics used during recording are stored with the applied ECG electrode location used during recording.

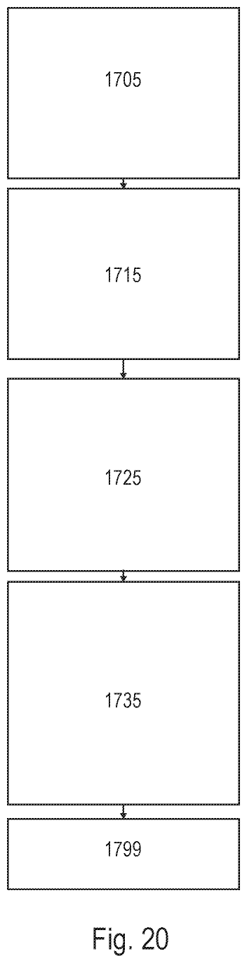

In step 1705 (FIG. 20), torso part tracking coordinates from step 1300 (1395) are retrieved. In step 1715, torso parts tracking coordinates are stored to local storage or connected storage. In step 1725, current recording ECG electrode locations are obtained from step 1300 (1395). In step 1735, currently recorded ECG electrode locations are stored to local storage or connected storage. The method ends in step 1799.

FIG. 21 shows six preferred embodiments 1, 2, 3 according to the present invention of the marker element. Marker element 1 consists of a blue rectangle allowing for identification based on at least the color and shape. The color allows for identification of the color and the meaning of such color as predetermined. Aspects of analysis of orientation with this embodiment will come from information in the 3-D image recording relating to e.g. the torso. Marker element 2 consists of a rectangle having two areas of color. Such rectangle also provides an analysis as to up and down and left and right sides based on the information of the colors. Therefore, more aspects of analysis can be determined based on the marker element in itself. Marker element 3 consists of a rectangle having a general element of color and a shape defined therein, in this case comprising a vertical line or bar with a circle generally oriented at the middle of the marker element 3.

In FIG. 21B, a further preferred embodiment of a marker element (30) according to the present invention is shown. The marker element comprises a generally diamond-shaped extension 32 that extends upwards from the main body of the marking element 30. This generally diamond-shaped extension is intended to be placed on the sternum

The marker element is a generally rectangular element comprising several visual elements that are usable for determining presence of a marker element in the imaging information of the torso. A first of those elements is the general shape itself. Another one of those elements is defined by four circles 34 in a generally trapezoidal shape. This shape both helps by recognizing the marker itself and is functional in defining lines that are functional at indicating parts of the torso at which ECG leads of features are to be expected.

A further embodiment is performed using an ECG system as input or an ECG system with features added thereto for embodying the invention. A typical embodiment comprises a computing device with receiving means for receiving from an ECG device the ECG measurements during an ECG session, such as during a procedure or for obtaining data to base a subsequent diagnosis on. The computing device is provided with a processor and memory. The memory comprises program code for enabling the processor to perform the method according to the invention.

Furthermore, the computing device is coupled to a monitor for displaying resulting images. A user interface is also displayed on the monitor for allowing input to be provided. Additional aspects of the user interface is comprised of a keyboard and mouse, touch screen, and all other user preferred in itself known input devices may be coupled to the computer through readily applicable connecting ports.

Furthermore, a 3-D camera is available for taking imaging information recordings from the torso. For obtaining the 3-D imaging information recordings, a capability to record from several sides of the torso is preferred. This is obtained by either one camera that is movable to capture images from the top, left and right side of the torso. Alternatively, two or more cameras may be fixedly mounted relative to the position of the torso in order to combine the 3-D imaging information recordings of the two or more cameras.

Furthermore, The computer is preferably connected to a database of 3-D torso models. Such a database of 3-D torso models preferably comprises unique torso models obtained by imaging devices, such as an MRI, CT or sound echo device. Depending on available time and equipment the respective information can advantageously be obtained during the ECG session, before the ECG session or based on historical measuring data for performing of this method.

Preferably, the 3-D photo is recorded by means of a 3-D camera providing a cloud of points in a 3-D space. The cloud of points represent the subject of the imaging information recording. To this end, the 3-D camera is used to capture an image of a torso of a subject in the form of 3-D information comprising information with respect to depth and color of the subject and of the surroundings of the subject. As indicated in the above, a single camera can be moved relative to the subject, such as along a generally circular line around the torso perpendicular to a longitudinal axis of the subject. Also multiple cameras can be used mounted around the subject for taking the appropriate recordings.

A main subject of the present invention is the use of a marker element to be used as a reference point relative to the torso. According to embodiments such a marking element provides an optically recordable element, such as a surface, perform an input for the analysis of the 3-D imaging information recording. And may take the form of a patch, optionally comprising communication electronics providing an identification, having predetermined recognizable characteristics for detecting thereof by means of the computing device executing the appropriate program means. Optionally, the computing device is partly or wholly integrated into the camera device.

Preferably, the position on the thorax is predefined and enables the computing device to match, orient and or detect the thorax in the 3-D imaging information recording under clinical circumstances. By applying the marker, according to the embodiment, computing device is unable to perform an analysis eliminating disturbances, such as blankets, equipment etc. Also, a quality check of the 3-D photo can be based on imaging information relating to the marker element.

Alternative embodiments of the marker element comprise recognition by means of color, signal, patterns, geometry, such as a shape.

The marker element provides a means to use as a basis for analysis. Algorithms of analysis are preferably adaptable to a range of predetermined marker times, such as distinguished by means of for example color, shape, dimension, lighting, sound. Preferably, the position of the marker element, or marker elements on the thorax is predefined, for example by having the upper side of the marker element coincide with the upper part of the sternum or supreasternal notch and having the marker element positioned along the sternum.

Several characteristics of several preferred embodiments of the marker element provide distinct advantages. Analysis of the color or combinations of color of the marker element provides advantages in permitting the detection of the marker element which enables analysis of an area of the subject where the marker is present. Providing a certain order of color them provide information regarding to orientation's such as left, right, top, bottom and depth orientations of the subject and allow for such information to be used as inputs in the analysis.

The geometry or shape of the marker element provides the advantage of improved performance of analysis, such as during detection of the marker on the subject.

Characteristics such as sound, light or a signal from an RFID chip provided in the marker element provides advantages in the direction of the marker and advantages in performing the analysis according to the present invention, such as in identifying the marker element on the thorax of the patients.

The marker element represents a reference point towards the algorithm performing the analysis in a way that defines the 3-D space independently of how the recording of the imaging information is performed. That is, independent of which camera is used, what the orientation of the camera is, on as the marker is comprised in the imaging information recording. The marker provides a basis for the algorithms to determine the orientation of the marker and based on that create an initial estimate of the orientation of the thorax. If non-preferable outcomes are obtained, information may be outputted as to a change in positioning of the camera are relative to the subject, such as to provide a better alignment with regard to e.g. a longitudinal axis of the torso, or to provide a better alignment relative to the marker element.

Analysis of the external shape of the subject is a further aspect in the marker element is set to improve. In case of e.g. a female subject, algorithms are provisions to detect the shape of a breast relative to the marker position. Based on this, specific analysis of the 3-D imaging information recording is performed. Advantages thereof are that the time required for performing calculations for the analysis is reduced allowing for a real-time usable results. As such, the marker element is preferably the starting point of the comparison between the 3-D imaging information recording and the 3-D model of the torso obtained.

An initial verification step in recording the 3-D imaging information recording comprises verification of the presence of the marker element. Preferably also a verification is made of acceptability of the image due to general photographic circumstances of the area or area in a room in which the recording is performed.

Furthermore, the 3-D imaging information recording is generated and verified with respect to the presence of the marker element, whereupon it is saved and used for further analysis.

FIG. 1 shows three preferred embodiments 1, 2, 3 according to the present invention of the marker element. Marker element 1 consists of a blue rectangle allowing for identification based on at least the color and shape. The color allows for identification of the color and the meaning of such color as predetermined. Aspects of analysis of orientation with this embodiment will come from information in the 3-D image recording relating to e.g. the torso. Marker element 2 consists of a rectangle having two areas of color. Such rectangle also provides an analysis as to the and down and left and right based on the information of the colors. Therefore, more aspects of analysis can be determined based on the marker element in itself. Marker element 3 consists of a rectangle having a general element of color and a shape defined therein, and his case comprising of a vertical line or bar with a circle generally oriented at the middle of the marker element 3.

FIG. 2 is provided to show an overview of a torso with a marker element on the sternum and ECG electrodes oriented on the torso. The quality of the ECG recordings, and comparability of a range of recordings over time is dependent on a correct orientation or the same orientation in the several recordings over time.

The present invention has as an important advantage that it becomes possible to relate the position of the ECG electrodes to the position of the marker element and thus to a fixed position on the torso. Furthermore, the present invention enables relating the imaging information recording to the model of the torso. Furthermore, the present invention enables relating the position of the marker element to the model of the torso, furthermore, the present invention enables relating the position of the ECG electrodes to the torso model, preferably wherein the marker element provides a basis for calculating such relation and allowing for calculating such relation very speedily, such as fast enough for providing a result usable within the session, defined as real-time within the context of the present invention.

In an embodiment of the analysis, the 3-D imaging information recording is divided in areas. A main area of analysis is the marker area 11. The marker area 11 is an area defined around a detected marker. Other areas comprise area has 16, 17, 18, which are area is defined to compare parts of the 3-D imaging information recording with the torso model information for reaching a match between those. The electrodes 13 are regular ECG electrodes, preferably recognizable by shape or color for identification thereof, which electrodes are to be matched to the torso model by means of imaging information and the presence of the marker therein. FIG. 3 provides a general overview of the method according to an embodiment. Initially the method is started in step 20. In step 21, imaging information as obtained from the 3-D camera is interpreted to assess the presence of a marker. It is preferred that imaging information is recorded in case such presence of a marker is assessed, in order to record usable information. In step 22, it is determined of a marker is present in imaging information. In case there is no marker detected in the imaging information, the method returns to step 21. In case a marker is determined to be detected in step 22 in the imaging information, the imaging information is structured to coordinates, color information and or depth. The result is a cloud of points in step 23.

In step 24, the results of step 23 are divided into areas of analysis, such as areas to be compared with areas of the 3-D torso model. In step 25 it is determined whether enough areas for further analysis are defined. In case it is determined that not enough areas for further analysis are defined, the method returns to step 21. In case it is determined that enough areas for comparison are defined, and as such a quality check of the 3-D imaging information recording provides a positive determination, the method continues in step 26.

In step 26, the preprocessed 3-D imaging information recording is matched to matchable information of the 3-D torso model as obtained. In step 27 it is determined whether a match was possible between the information from the 3-D imaging information recording and the 3-D torso model. In case it is determined that the match was not possible, the method returns to step 21 in order to reprocess with a new 3-D imaging recording. In case it is determined that the method provided a match of an acceptable quality, such as within certain predetermined limits, the electrodes are detected from the 3-D imaging information and matched with the torso model for adding information relating to the electrodes to the 3-D torso model.

FIG. 4 provides a further embodiment of the method according to the present invention. The method starts in step 100 as a configuration step 4 loading resources needed, based on an instruction into the user interface for generating a 3-D image recording. The detection algorithm is initialized by providing characteristics of the marker element used in the respective session. Those characteristics, such as colors, such as blue, green or pink, geometry, such as rectangular, square, triangular, or its dimensions, such as with or height, are retrieved from the database. These characteristics I used for creating groups of points having at least one of the marker description, such as the color.

In step 110, the information relating to the 3-D imaging information is received from the 3-D camera comprising e.g. color, depth and etc. The information is structured to coordinates having color information.

In step 120, the 3-D imaging information is prepared for analysis for detecting the marker the received imaging information is analyzed for the presence of the marker. The marker has to be oriented in the marker area 11 that is displayed, preferably yellow, on a display of the camera or a monitor of the computing device showing the imaging information. Pixels that are received inside the marker element area 11 is added to a list with the same criteria. A list is created for each criterion. This helps in finding the marker as pixels with the same color, such as on the marker will be on the same list.

In step 130, the marker is identified based on the created lists. Information as to the geometry of the marker is extracted from the information of the pixels in the lists. If this is not successful, a calibration is performed with respect to constraints such as circumstances such as light in the room that can influence the colors of the recording. If the marker is found, the method allows in step 140. An example is a marker that is a rectangle, one half centimeter wide, 5 cm high and having a blue-collar. The step of analyzing will identify the list having the pixels providing the rectangular shape, such as by taking 4 points of the selected list and calculating the angle created by each 3 points. If the angle is 90°, the distance and the color match, and then the list comprises information relating to the marker.

In step 140, the order of calibration and a zone of calibration is processed. If the marker was not detected, the camera is directed such that the marker is in the marker element area 11. The said calibration is performed and step 130 is repeated. In step 150, it is determined that the 3-D imaging information contains the marker and the 3-D imaging information is recorded. One session according to the present invention may comprise a number of recordings over the duration of the ECG session.

Based on the created 3-D imaging recordings, a full 3-D imaging recording of the torso is created. Images are taken of the torso from several angles, all comprising the marker. For example, the camera is moved by starting capturing from the left part of the torso moving over the torso to the right part of the torso. During such movement of the camera the camera takes a temporary image recording every second after which the recordings are combined to a 3-D image recording of the full torso. All individual recordings are processed as described in the above in order to assess the presence of the marker.

In step 170, the 3-D image recording of the full torso is verified. In case the combination of the sip recordings electrodes to deformations you to inconsistent camera moving, the recordings have to be taken again by repeating the above steps.

In step 180, it is determined if the resulting marker from the combination of separate 3-D imaging information recordings is valid. Such validity is obtained if the combined 3-D image recording comprises sufficient information, such as for example performed by analyzing the 3-D imaging information recording starting from the marker position down to the bottom by steps of 3 cm and checking the percentage of holes present in the 3-D photo. Acceptability is for instance defined if the percentage is below 3%. In step 190, the method ends with outputting a validated 3-D photo

FIG. 4 describes an embodiment of pre-analysis in preparation for computation during analysis. This phase represents a pre-analysis of this 3-D imaging information recording for extracting information relating to the subject, was information is of assistance in the correction of errors in the 3-D imaging information, the recognition of parts of the subject such as the shoulders, head, breast areas. The purpose of this embodiment is to analyze usability of available information for the extraction of the thorax information relating to the electrode positions. In step 200, the method is initialized by loading the 3-D imaging information and analyzing means, such as a tracker for shoulders. Here, the characteristics of the marker is related to a specific use. A blue marker is for instance used for analyzing the anatomy of the body, such that for instance if the marker is rectangular and its color is plain blue the analysis of the body will be performed relating to the circumference of the torso the width or the circumference of the arm.

Step 210 comprises loading or detection an anatomical thorax element/parts from the 3D-Photo and categorization those element/parts relating them to their body positions. The detection in this level is a categorization per region relative to the marker position as depicted in FIG. 9.

A coordinate analyzer separates the points of 3-D imaging information to points which are in higher position than the position of the marker then divide this upper part to left and right category. The other points from the 3-D imaging information, preferably having an altitude lower than the marker's altitude, those will be in the list (group) representing the belly.

The detection of the thorax is a combination of information given by specific analyzers. A shoulders analyzer will give the position of the shoulders, a circumference analyzer gives us different circumferences related to an altitude (horizontal position). From those two information we have the upper offset of the thorax and also the left and right is deduced from the ellipse equation of the biggest circumferences found.

Step 220 is defining the shape of the shoulders, for example using a cylindrical geometry approach, such as shown in FIG. 10. The shoulders analyzer gets lists of points and studies the relations thereof toward each other. In other words, the analyzer search for area where the depth of points decreases progressively for those points to be in the same distance from a line, such as a central axis of the cylinder they form.

In step 230, the circumference of the torso is analyzed. For the detection of the circumference of the torso, the symmetry and the variation in the distance of the torso in the same altitude is analyzed. Curved lines are created with points at the same altitude at for instance a 2 cm interval. Ellipses are created in this way for determining the circumference of the torso for permitting the prediction of the dimension of the subject. This prevents manipulation reduction.

In step 240, other characteristics of the torso are determined, such as the head, hair, or skin color. Also elements in the photo that are distracting to analyzing the torso, such as a blanket on the subject, are rejected as much as possible. Preferably, it is known what color blanket is used such that imaging points having the same color as the blanket can be removed from analysis, thereby saving time of analysis by such noisy information.

In step 250, the information from the analyzer is from the steps 220, 230, and 240 are combined to improve the analysis. For example, by knowing the position of the shoulders and the points in the imaging information constituting the shoulders, the central axis of the cylinder that they form helps in detecting borders where the imaging information may end, for example because there are no electrodes beyond such areas.

FIG. 6 shows a method for matching the 3-D torso model to the 3-D imaging information recording. The method starts in step 500 with loading information, and initializing the computing device for performing the calculations. The input of step 510 is the information relating to the 3-D imaging information recording and the 3-D torso model. The position of the marker element in the imaging information recording as well as its equivalent position in the 3-D torso model is taken as the basis to calculate the difference of distance and the angles created by the kerf of the marker zone in the model and the imaging information.

Step 510 provides: take the marker position and its equivalent position by the model, then calculate the difference of distance and the angles created by the curve of the marker zone in the model and 3D photo.

The coordinate of the marker initially in the 3D photo is expressed in camera space (is the camera which give the point of 3D photo its coordinate) and the marker coordinate in the model is defined by the MRI device as consequence, except coincidence, the position of the marker in the 3D photo and model are different. The positions of the marker and the equivalent of the marker are separate by a distance not nul 0, see FIG. 12.

The normal in the position of the marker in 3D photo and in the model are the same. That is why the angles between the should be nul as consequence transformation has to reduce that angle to 0, see FIG. 11.

Step 520 provides: the 3D-Photo is moved to the model until the distance of the marker and its equivalent position is reduced to 0.

The distance between the marker in 3D photo and its equivalent position in the model is calculated then the 3D photo is translated to the position of the marker. Indeed we calculate the vector of translation and we move the coordinates of every point of the 3D photo using the calculated vector.

### EXAMPLE

A—translate—>B

A+vector=B

Vector=B−A.

See FIG. 2 and FIG. 13

Step 530 provides: 3D-Photo will be rotated until their curves are positively parallel and control if the distance between the marker and its equivalent position is null, see FIG. 14.

After translating the 3D photo to model the normal vectors (representation of the curve) of the model and the 3D photo must have an angle equal to 0 degree. If is not the case points of 3D photo are rotated until that angle becomes null. See FIG. 11

Step 540 provides: the new update of 3D-Photo is present but that does not grant that the photo and the model are in the same orientation.

This phase permits the correction of orientation of the 3D-Photo and the model by taking two equivalent random zones and calculation of two vectors which are going from the marker position to that area and rotate the 3D-photo until that two points coincide or become equal, see FIG. 12 and FIG. 15.

Step 550 provides: Estimation of the matching is the calculation of the difference of distance between projection of the element of 3D-Photo and their equivalent from the model.

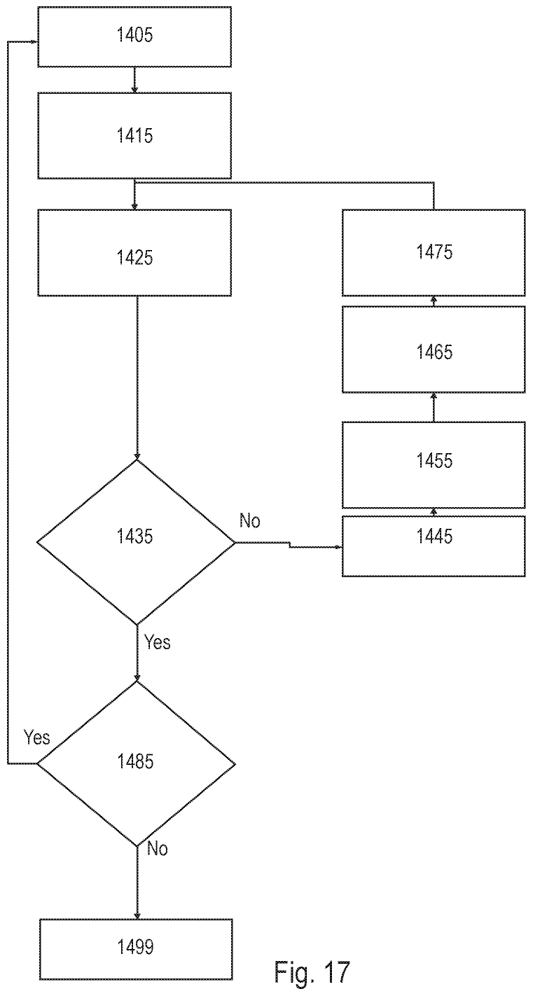

The following configuration of matching the marker position and a second point which is in 180 millimeter from the marker position is considered. We select a list of second point (random point) which are in same distance from marker in 3D photo then we calculate the transformation every time we select a point from the list of second point. Then the percentage of the difference of distance of every point from 3D photo and its projection into model is calculated and the best percentage is taken after comparing all transformations there are. See FIG. 13, FIG. 16 and FIG. 17.

Step 560 provides: compare with latest estimation if the value increased or not and apply the best transformation. If the percentage increased the calculation will be retried with another configuration until a maximum related to the model and the 3D-Photo is reached.

After step 550 the best percentage relative to the selected list of second point (point from random zone) is obtained. If the current best percentage is better than the old one the estimation improved.

Every time there is improvement the process is reiterated until that every percentage resulting from a selected configuration stays lower that the saved one.

By comparing every possibility it is indicated that the best result that can be provided with that 3D photo is obtained, see FIG. 15, FIG. 13 and FIG. 18.

Step 570 provides: the new 3D-photo is stored and can be used to extract the specific information and apply them to the model like electrodes position for example.

FIG. 7 shows a method for analyzing areas on the imaging information recording and defining a coordinate system relative to the marker element.

Step 600 provides: load the 3D-Photo and load the resources needed for the process (load the specific analyzer).

Step 610 provides: the marker analyzer goes to position of the marker and use the information related to it and creates from those information the “marker” to which every data is related. The points of the 3D photo in the space (coordinates) are positioned related to that marker, see FIG. 19.

The marker contains 3 axes to which every coordinate of a points from the 3D photo is expressed, see FIG. 20.

A point is taken from the 3D photo. That point after recording is expressed with the coordinate that the camera gives to that point in one side. In the other one, the coordinate of a point from the model is expressed with the coordinates given by the MRI device. The marker in 3D photo and its equivalent in the model are the same. As consequence, the coordinates are expressed using the same reference.

Step 620 provides: by the start of analyzing using the information generated from “610”, the zone of analysis is defined, see FIG. 20.

The zone of analysis is the result of the combination of the information that the analyzers provide. The line of the shoulders is the upper border of analysis. The radius of the biggest circumference plus the ellipse's center of all circumferences provides the offset left and right from the axis formed by the ellipse's center.

Step 630 provides the nearby area (or zone of the analysis) is inspected and is prepared to be categorized to the zone marker or random zone.

An nearby area or the marker zone is the area related to list of points which are close to the marker with a certain distance (3 cm, 5 cm, . . . ). All elements outside of the marker zone are in the random zone or what is called the control zone, see FIG. 14.

The analyzer calculates the curve of the geometry where the marker is positioned. The curve is how the concavity of this zone of the 3D-photo behave, see slide 6

The objective is to find the relation of the points and how the global curvature of the area behaves (manifest) due to the repartition. The result of the analysis of the curve is a normal vector that characterizes the area. For example if the area is plane then the normal (perpendicular vector) of the plane is a representation of the curvature which not changes along that plane. If the area is spherical then the normal is perpendicular to the tangent plane of the sphere in that selected position.

The analyzer get the no marker zone (random area), get their characteristics and store them to use them in matching process. The areas are characterized by their distances, their positions, see FIG. 19, see step 630.

The end analyzing structures the obtained information and store those to use those in further process.

The objective is to have 3D points with the same characteristic from the model and their equivalent in 3D photo (3D point from the model which is 80 millimeter far from the marker) to compare every points and with its equivalents to control the quality of matching, see FIG. 15.

Combine the marker with information and correct if there are errors present. The present invention has been described in the foregoing on the basis of several preferred embodiments. Different aspects of different embodiments are deemed described in combination with each other, wherein all combinations which can be considered by a skilled person in the field as falling within the scope of the invention on the basis of reading of this document are included. These preferred embodiments are not limitative for the scope of protection of this document. The rights sought are defined in the appended claims.

1. A method, such as implemented on a computing device part of and/or coupled to an ECG device, for providing an ECG analysis interface relating to a person, the method comprising steps of:
obtaining data entry identifying the person for obtaining historical data pertaining to at least one preceding data point from a database,
obtaining at least one historical ECG measurement result pertaining to said at least one data point from the database,
obtaining historical ECG electrode location information relating to the electrode placement of the respective at least one data point,
obtaining latest or new ECG electrode location information measurement of electrode positions for a live ECG measurement,
obtaining latest or new ECG measurement results of the person with the location measured electrodes,
performing steps of verification as to differences between the historical ECG electrode location information and the latest or new ECG electrode location information,
assembling the ECG analysis interface comprising a representation of differences between the at least one historical ECG measurement result and the latest or new ECG measurement results, preferably if the steps of verification provide a sufficient result.

2. The method according to claim 1 in which the representation of differences comprises a waveform representation of the historical ECG measurement results and of the new ECG measurement results.

3. The method according to claim 2 comprising steps of aligning the respective waveform representations, preferably aligning the respective waveform representations in relation to a time axis of an ECG graph.

4. The method according to claim 2 comprising steps of aligning the respective waveform representations based on respective RMS signals.

5. The method according to claim 3 in which the alignment is based on the peak of the QRS complex, preferably based on the peak of the QRS complex according to the RMS signal.

6. The method according to claim 1 providing the ECG analysis interface, preferably representation of differences thereof, with highlighting information based on a predetermined set of criteria.

7. The method according to claim 1 in which the predetermined criteria are based on a recognition of relevant differences relating to at least one condition or ailment.

8. The method according to claim 1 in which the at least one condition or ailment comprises a arrhythmogenic cardiomyopathy (ACM), sudden cardiac death risk (SCD), arrhythmogenic right ventricle cardiomyopathy (ARVC), initial stages of PVC development.

9. The method according to claim 1 in which the representation of differences comprises at least one numerical representation of the differences, preferably thereby indicating at least one respective biomarker.

10. A method, such as implemented on a computing device part of and/or coupled to an ECG device, for providing an ECG analysis interface relating to a person, the method comprising steps of:
obtaining data entry identifying the person for obtaining historical data pertaining to at least one preceding data point from a database,
obtaining historical ECG electrode location information relating to the electrode placement of the respective at least one data point,
obtaining an optical, preferably 3D, recording of the torso of the person and/or latest or new ECG electrode location information measurement of an initial placement of positions of the ECG electrodes for a live ECG measurement,
rendering or assembling feedback information for providing a feedback signal based on the historical ECG electrode information of the respective at least one data point,
visualizing of the feedback signal relative to the torso of the person.

11. (canceled)

12. The method according to claim 10 in which the steps of visualizing of the feedback signal relative to the torso of the person comprises steps of at least one of the following:
visualizing the feedback signal relative to the torso in an augmented reality device;
visualizing the feedback signal relative to the torso by projecting the feedback signal on the torso by at least one projector, such as a video beamer;
visualizing the feedback signal relative to the torso by displaying the torso with a projection of the feedback signal on a display monitor;
visualizing the feedback signal relative to the torso by projecting the feedback signal on the torso by means of light spots from a laser projector.

13. The method according to claim 10, in which the location information comprises 3D location information as measured with at least one 3D camera.

14. The method according to claim 10 comprising steps of receiving patient information, such as a patient history comprising person characteristics relating to the at least one historical ECG measurement result and/or the hysterical ECG electrode location information.

15. The method according to claim 10 in which the steps of obtaining historical ECG electrode location information comprises steps of obtaining historical torso shape information, preferably in which the steps of obtaining the latest or new ECG electrode location information comprises steps of obtaining latest or new torso shape information.

16. The method according to claim 15 comprising steps for verifying that the historical data pertains to the person, such as by comparing differences between the historical torso shape and the latest or new torso shape.

17. The method according to claim 15 in which the steps of assembling a graphical representation signal for displaying differences between at least one historical torso shape and the latest or new torso shape.

18. The method according to claim 10 in which the steps of receiving patient information comprises steps of requesting further patient information based on a patient identification, such as a person identification number, weight and/or height.

19. The method according to claim 10, comprising steps of:
comparing hysterical ECG electrode location information of the at least one data point with the new ECG electrode location information,
rendering or assembling feedback information for providing a feedback signal as to an incorrect new ECG electrodes location, preferably with an indication of the correct location.

20. The method according to claim 10, comprising steps of obtaining latest on new ECG measurement results of the person with the location measured electrodes.

21. The method according t claim 10, in which the steps of obtaining ECG electrode location information comprises steps of obtaining an optical, preferably 3D, recording of the torso of the person with latest or new ECG electrode locations of a placement position of the ECG electrodes.

22. The method according to claim 10 comprising steps of taking for differences or similarities between a respective historical torso shape and the latest or a new torso shape.

23. A system for application of a method claim 1, comprising:
a processing unit,
a memory coupled with the processing unit,
receiving means for receiving data entry identifying a person,
receiving means for receiving historical ECG electrode location information,
receiving means for receiving latest or new ECG electrode location information,
outputting means for outputting a feedback signal based on the historical ECG electrode information of the respective at least one data point.

24. A system for taking recordings, such as 3D imaging recordings, of a body or a torso thereof, preferably in conjunction with a method according to claim 1, comprising:
a processing unit,
a memory coupled with the processing unit,
receiving means for receiving data entry identifying a person,
receiving means for receiving historical ECG electrode location information,
receiving means for receiving latest or new ECG electrode location information,
outputting means for outputting a feedback signal based on the historical ECG electrode information of the respective at least one data point.

25. (canceled)
